SEQUENCE LISTING (2 sequences)

SEQ ID NO: 1 - protein, 10 aa, Artificial Sequence
  SYNTHETIC CONSTRUCT
IAYPYDVPDY

SEQ ID NO: 2 - protein, 8 aa, Artificial Sequence
  SYNTHETIC CONSTRUCT
YPYDVPDY

(19) United States
(12) Patent Application Publication
(10) Pub. No.: US 2020/0023054 A1
(43) Pub. Date: Jan. 23, 2020

(54) DEVELOPMENT OF AN ALTERNATIVE MODIFIED LIVE INFLUENZA B VIRUS VACCINE

(71) Applicant: UNIVERSITY OF GEORGIA RESEARCH FOUNDATION, INC., Athens, GA (US)

(72) Inventors: Jefferson J.S. Santos, Athens, GA (US); Daniel R. Perez, Athens, GA (US)

(21) Appl. No.: 16/496,049

(22) PCT Filed: Mar. 21, 2018
(86) PCT No.: PCT/US2018/023563

§ 371 Date: Sep. 20, 2019

Related U.S. Application Data
(60) Provisional application No. 62/474,604, filed on Mar. 21, 2017.

(51) Int. Cl. A61K 39/145; A61P 31/16

(52) U.S. Cl. CPC A61K 39/145; A61K 2039/5254; A61P 31/16

(57) ABSTRACT

Disclosed are compositions and methods related to live attenuated Influenza B viruses.

This application claims the benefit of U.S. Provisional Application No. 62/474,604, filed on Mar. 21, 2017, which is incorporated herein by reference in its entirety.

This invention was made with government support under Grant No. HHSN266200700010C and HHSN272201400008C awarded by the National Institutes of Health. The government has certain rights in the invention.

### I. BACKGROUND

Influenza B virus (IBV) is an envelope virus with a negative-sense, segmented, single-stranded RNA genome in the Orthomyxoviridae family Eight viral RNA (vRNA) segments are present in the IBV genome encoding at least 11 proteins. IBV is considered a major respiratory pathogen of humans with a well-documented history of epidemics. Although IBV infects all age groups, it causes substantially more disease burden in the very young and the elderly. In the United States, in each season between 2004 and 2011 (excluding the 2009 pandemic), 22-44% of all pediatric influenza-related deaths were associated to IBV infections. In the United States and Europe, epidemiological evidence in recent years reveals the burden of IBV is potentially increasing. Phylogenic studies showed the emergence of two distinct IBV lineages that diverged in the 1970s whereas serological evidence in the 1980s revealed that these lineages have become antigenically distinct. These two lineages are known as Yamagata (B/Yam) and Victoria (B/Vic) lineages, with virtually no serum cross-reactivity against each other when evaluated by hemagglutination inhibition (HI) assay. Although the mutation rate is lower than observed with IAV, both IBV lineages continue to undergo antigenic drift as a result of the error prone characteristics of the viral polymerase and host-mediated antibody pressure.

Vaccines against seasonal influenza viruses are manufactured to confer protection against IAV and IBV viruses. For protection, current vaccines rely primarily on antibody response to the hemagglutinin (HA) viral surface protein. Antigenic drift of HA requires influenza vaccines to be updated regularly to antigenically match the currently circulating strains. Seasonal influenza vaccines traditionally possessed three influenza virus strains, including two IAV strains (A/H1N1 and A/H3N2) and one IBV strain, representing either B/Yam or B/Vic lineages. In recent years, however, the two IBV lineages have shown not only seasonal variations but also significant differences in prevalence within different countries making it extremely difficult to predict which IBV lineage would be predominant in a season in a particular region. Thus, significant antigenic mismatch between seasonal vaccines and circulating IBV strains have been reported in various parts of the world. In response to this effect, quadrivalent vaccines have been approved and are available that incorporate both IBV antigenic lineages in addition to the two IAV strains.

Licensed seasonal influenza vaccines are available in the United States as inactivated influenza vaccines (IIV), recombinant influenza vaccines (rIV) or live attenuated influenza vaccines (LAIV). LAIV vaccines are produced using master donor viruses (MDVs) that carry a series of mutations that restrict virus replication to the upper respiratory tract (absent or reduced lower respiratory tract replication and minimal clinical signs). In the US, MDVs were produced by Maassab et al for IBV and IAV by serial passage of the B/Ann Arbor/1/66 (MDV-B) and A/Ann Arbor/6/60 (H2N2) (MDV-A) viruses, respectively, at progressively lower temperatures, resulting in cold-adapted (ca), temperature sensitive (ts), in vivo attenuated (att) viruses that grew well at 25° C. Although both MDV-B and MDV-A strains show many mutations compared to their respective parental viruses, those that impart the ca/ts/att phenotype are located primarily in the polymerase-complex (PB1, PB2, PA, and NP). The ca/ts/att mutations in MDV-B were mapped to PB2 (S630R), PA (V341M), NP (V114A, P410H and A509T) and M1 (H159Q and M183V), whereas those in MDV-A lie within PB2 (N265S), PB1 (K391E, D581G and A661T) and NP (D34G). These strains have been commercially available under the trade name FLUMIST in the United States following approval by the Food and Drug Administration (FDA) in 2003. However, FLUMIST has suffered a number of issues in vaccine effectiveness over the past three seasons (2013-2014, 2014-2015, and 2015-2016), highlighting the critical importance of continued investment in LAIV development in order to develop more efficacious vaccines.

### II. SUMMARY

Disclosed are methods and compositions related to live attenuated Influenza B viruses.

In one aspect, disclosed herein are live attenuated influenza B viruses comprising a substitution at residue 580 and/or 660 of the PB1 segment of the viral polymerase.

Also disclosed are live attenuated Influenza B viruses of any preceding aspect, wherein the substitution at residue 580 is a E580G substitution.

Also disclosed are live attenuated Influenza B viruses of any preceding aspect, wherein the substitution at residue 660 is a S660A substation.

In one aspect, disclosed herein are live attenuated influenza B viruses comprising an E580G and/or a S660A substitution in the PB1 segment of the influenza viral polymerase.

Also disclosed are live attenuated Influenza B viruses of any preceding aspect further comprising a HA-tag.

In one aspect, the live attenuated influenza B virus of any preceding aspect can be a component in a vaccine.

In one aspect, disclosed herein in are live attenuated influenza vaccines of any preceding aspect wherein the vaccine is a quadrivalent vaccine further comprising gene segments from influenza A viral strains H3N2 and H1N1.

Also disclosed herein are methods of inhibiting and/or preventing an Influenza B virus infection comprising administering to a subject a live attenuated Influenza B virus of any preceding aspect or a vaccine comprising said live attenuated Influenza B virus.

In one aspect, disclosed herein are methods of attenuating an influenza B virus comprising substituting the glutamate at residue 580 and/or the serine at residue 660 of the PB1 segment of the viral polymerase. In one aspect, the attenuation can occur by substituting the nucleic acids that encode for the glutamate at residue 580 and/or the serine at residue 660 for nucleic acids encoding other amino acids.

Also disclosed are methods of attenuating an influenza B virus of any preceding aspect, wherein the glutamate at residue 580 of the PB1 segment of the viral polymerase is substituted with a nonpolar amino acid, including, but not limited to alanine, glycine, valine, leucine, or isoleucine.

Also disclosed are methods of attenuating an influenza B virus of any preceding aspect, wherein the serine at residue 660 of the PB1 segment of the viral polymerase is substituted with a nonpolar amino acid, including, but not limited to alanine, glycine, valine, leucine, or isoleucine.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows a schematic representation of the IBV genome organization depicting all the modifications tested for IBV attenuation and their respective outcomes. Site-directed mutagenesis and inverse PCR were used to incorporate the attenuation modifications into the PB1 (E391, G580, A660 and HA tag) and PB2 (S267, F359 and Y406) gene segments. The PB1 E391 modification was unstable. The PB2 S267 was deleterious for virus rescue while the PB2 F359 and PB2 Y406 substitutions increased IBV virulence. G580, A660, and HA tag modifications into the PB1 segment were stable and sufficient to confer attenuation (PB1 att).

FIGS. 2A and 2B show that mutations in PB2 increase IBV virulence. The PB2 F406Y and PB2 W359F mutations were individually introduced into the PB2 segment of the B/Brisbane/60/2008 strain by site-directed mutagenesis and used for virus rescue using reverse genetics. Female 6-week-old DBA/2J mice wereinoculated I.N. with 10⁵ EID50 of the WT RG-B/Bris, B/Bris-F406Y, and B/Bris PB2-W359F viruses. The mock group received PBS and served as negative control. (2A) Percentage of change in body weight and (2B) survival rate following I.N. inoculation were monitored daily. Plotted data represent means±standard error (SD). Two-way analysis of variance (ANOVA) was performed to calculate P values. ***, P<0.001.

FIGS. 3A, 3B, and 3C show the characterization of the IBV PB1 modifications in vitro. FIG. 3A shows the HA tag expression in the IAV and IBV viruses carrying a chimeric PB1HA protein. MDCK cells were 1) mock-inoculated (PBS) or inoculated with the following strains: 2) WT RG-B/Bris, 3) B/Bris ts, 4) B/Bris att or 5) IAV att control (7attWF10:1malH7). PB1HA chimeric proteins with molecular weight (MW)>80 KDa are shown: IAV-PB1HA is indicated by a black arrow (predicted MW 88.66 KDa) and IBV-PB1HA is indicated by an open arrow (predicted MW 85.35 KDa). The host cellular protein glyceraldehyde-3-phosphate dehydrogenase (GAPDH, 38.5 KDa) is shown as gel loading control. FIG. 3B shows the virus polymerase activity at different temperatures. 293T cells were transfected with plasmids encoding the PB1 (or PB1 att), PB2, PA, NP and a IBV vRNA influenza virus-driven luciferase reporter replicon. In addition, a plasmid encoding the secreted alkaline phosphatase under the control of the CMV promoter was co-transfected to normalize variations in transfection efficiency). Relative polymerase activity was calculated as the ratio of luciferase activity to alkaline phosphatase activity at 24 or (3C) at 48 hpt. Plotted data represent means±standard error (SD). Two-way analysis of variance (ANOVA) was performed to calculate P values. **, P<0.01; ****, P<0.0001.

FIGS. 4A, 4B, 4C, 4D, and 4E show that B/Bris att virus displays ts phenotype in growth kinetics in vitro. Confluent monolayers of MDCK cells were inoculated at 0.01 MOI with either WT RG-B/Bris or B/Bris att viruses and incubated at 33 (4A), 35 (4B), 37 (4C), 37.5 (4D), and 39° C. (4E). At the indicated time points, tissue culture supernatant from inoculated cells was collected for quantification of virus titers by TCID₅₀ using the Reed and Muench method. Plotted data represent means±standard error (SD). Two-way analysis of variance (ANOVA) was performed to calculate P values. *, P<0.05; **, P<0.01; ***, P<0.001; ****, P<0.0001.

FIGS. 5A, 5B, 5C, 5D, 5E, 5F, and 5G show the safety and immunogenicity of the B/Bris att virus. Female 6-week-old DBA/2J mice were inoculated I.N. with 10⁶ EID of either WT RG-B/Bris or B/Bris att viruses. The mock-inoculated group received PBS and served as a vaccine negative control. FIG. 5A shows the percentage of change in body weight of mice was monitored daily following I.N. inoculation with the B/Bris att virus. FIG. 5B shows the virus replication and tissue tropism of the WT RG-B/Bris and B/Bris att viruses in the respiratory tract of mice. At 3 and 5 dpi, four animals from each group were euthanized, and virus titers in the upper respiratory tract (nasal turbinates, NT) or (5C) lower respiratory tract (lungs) of mice were determined by standard TCID₅₀ in MDCK cells. Plotted data represent means±standard error (SD). Two-way analysis of variance (ANOVA) was performed to calculate P values. *, P<0.05; ***, P<0.001. Lungs from mice euthanized 5 days post-vaccination were collected and preserved in 10% formalin for histopathological examination by H&E staining Images were taken at 20× magnification. Lung H&E staining from (5D) mock-inoculated (PBS) mice, (5E) B/Bris att-vaccinated mice, (5F) WT RG-B/Bris-inoculated mice. FIG. 5G shows the immunogenicity of the B/Bris att virus in mice sera measured by HI assay against the homologous (B/Bris) and heterologous (B/Wis) viruses at 21 dpi.

FIGS. 6A, 6B, 6C, 6D, and 6E show the protective efficacy of the B/Bris att virus against challenge with B/Bris PB2-F406Y virus. Female 6-week-old DBA/2J mice were I.N. inoculated with PBS (mock-vaccinated negative control) or inoculated with 10⁶ EID50 of the B/Bris att virus. Three weeks post-inoculation, mice were challenged with either PBS (mock) or 10⁷ EID50 of the B/Bris PB2-F406Y virus by the I.N. route. (6A) Survival rate and (6B) percentage of change in body weight following challenge with the B/Bris PB2-F406Y virus was monitored daily. FIG. 6C shows virus replication and tissue tropism of the B/Bris PB2-F406Y virus in the respiratory tract of vaccinated (B/Bris att) or negative control (mock-vaccinated) mice after challenge. At 3 and 5 dpc, four animals from each group were euthanized, and virus titers in the upper respiratory tract (nasal turbinates, NT) or (6D) lower respiratory tract (lungs) of mice were determined by standard TCID₅₀ in MDCK cells. FIG. 6E shows the serum antibody response in mice measured by HI assay against the homologous (B/Bris) and heterologous (B/Wis) viruses at 21 dpc. Plotted data represent means±standard error (SD). Two-way analysis of variance (ANOVA) was performed to calculate P values. **, P<0.01; ****, P<0.0001.

FIGS. 7A, 7B, 7C, 7D, and 7E show the protective efficacy of the B/Bris att virus against challenge with antigenically heterologous B/Wis PB2-F406Y virus. Female 6-week-old DBA/2J mice were I.N. inoculated with PBS (mock-vaccinated negative control) or inoculated with 10⁶ EID₅₀ of the B/Bris att virus. Three weeks post-inoculation, mice were challenged with either PBS (mock) or with 10⁷ EID₅₀ of the B/Wis PB2-F406Y virus by the I.N. route. (7A) Survival rate and (7B) percentage of change in body weight following challenge with the B/Wis PB2-F406Y virus was monitored daily. FIG. 7C shows the virus replication and tissue tropism of the B/Wis PB2-F406Y virus in the respiratory tract of vaccinated (B/Bris att) or negative control (mock-vaccinated) mice after challenge. At 3 and 5 dpc, four animals from each group were euthanized, and virus titers in the upper respiratory tract (nasal turbinates, NT) or (7D) lower respiratory tract (lungs) of mice were determined by standard TCID₅₀ in MDCK cells. FIG. 7E shows the serum antibody response in mice measured by HI assay against the homologous (B/Bris) and heterologous (B/Wis) viruses at 21 dpc. Plotted data represent means±standard error (SD). Two-way analysis of variance (ANOVA) was performed to calculate P values. *, P<0.05; **, P<0.01; ****, P<0.0001.

FIGS. 8A, 8B, 8C, 8D, and 8E show that B/Bris att vaccination reduces lungs pathology after IBV challenge. Lungs from mice euthanized at day 5 post-challenge were collected and preserved in 10% formalin for H&E staining. Images were taken at 20× magnification. Lung H&E staining from (8A) mock-vaccinated mice following challenge with B/Bris PB2-F406Y (8B) B/Bris att-vaccinated mice following homologous challenge (B/Bris PB2-F406Y), (8C) mock-vaccinated, mock-challenged (PBS) control mice, (8D) mock-vaccinated mice following challenge with B/Wis PB2-F406Y and (8E) B/Bris att-vaccinated mice following heterologous challenge (B/WisPB2-F406Y).

FIG. 9 shows that the QIV vaccine was effective against aggressive challenge with influenza A and B viruses. 100% survival was observed in QIV-vaccinated mice after challenge with either B/Bris PB2 F406Y virus or mouse-adapted Ca/04 (H1N1) virus. Five to six-week old female DBA/2J mice (The Jackson Laboratory, Bar Harbor, Me.) were used in all mice experiment. Each mouse was anesthetized with isoflurane and was subsequently inoculated intranasally (i.n) with 50 μl of either the QIV formulation (n=22) or mock vaccinated PBS control (n=33). The QIV formulation contained live attenuated influenza A and B viruses (Ty/04 att H3N2, Ca/04 att H1N1, B/Bris att and B/Wisc att) at a dose of 10⁶ TCID₅₀ each virus/50 μl per mouse. At 20 day-post-inoculation (dpi), mice were bled from the submandibular vein and sera collected to measure neutralizing antibody responses (4 mice/group). At 21 dpi, the mice were boosted using the QIV as explained above. The day before challenge [20 day-post-boost (dpb)], 4 mice/group were bled and sera collected to measure neutralizing antibody responses. At 21 dpb, mice were challenged with either 10⁷ TCID₅₀/mouse of mouse-adapted Ca/04 (ma-Ca/04) (QIV group n=11, mock vaccinated PBS n=11) or with 10⁷ TCID₅₀/mouse B/Bris PB2-F406Y (QIV group n=11, mock vaccinated PBS n=11). A third group was challenged with mock PBS as controls (n=8). The mice were monitored daily for body weight loss and survival. At 5 day-post challenge (dpc), 3 mice per group were sacrificed and lung and nasal turbinate samples were collected for quantitation of virus titers. At 21 dpc, all mice were sacrificed and sera, nasal washes and bronchoalveolar lavage fluids (BALFs) were collected to evaluate antibody responses.

FIG. 10 shows that at 5 day-post challenge (dpc), 3 mice per group were sacrificed and lung and nasal turbinate samples were collected for titration of virus shedding.

FIG. 11 shows serum samples collected at 20 dpi, 20 dpb and 21 dpc were analyzed for the presence of neutralizing antibodies by the hemagglutination inhibition (HI) assay using the WT Ty/04 (H3N2), ma Ca/04 (H1N1), WT B/Bris and WT B/Wis viruses (9). Briefly, mice sera were treated with receptor destroying enzyme overnight 37° C., and then heat inactivated at 56° C. for 30 min. Then, the sera were diluted 1:10 with PBS and subsequently serially diluted 2-fold and mixed with 8-hemagglutination units (HAU) of virus for 15 min at room temperature. HI activity was monitored by adding 0.5% turkey red blood cells to the virus antibody mixtures. Plates were read after 30 min at room temperature.

FIG. 12A shows that QIV-vaccinated mice showed high levels of IgA antibody against the ma Ca/04 (H1N1) virus noticeable at 5 dpc (compared to the PBS control challenge with ma Ca/04 (H1N1) that shows no IgA responses). IgA responses are maintained in mice for at least 3 weeks after challenge. Virus specific IgA antibodies were detected by enzyme-linked immunosorbent assay (ELISA) using samples from lungs and nasal turbinates collected at 5 dpc and from feces, sera, nasal washes and BALF collected at 21 dpc. ELISA plates were coated with ma-Ca/04 virus overnight at 4° C. The plates were blocked with 10% skim milk in PBS for 2 h at room temperature and then washed with PBS containing 0.05% Tween 20 (PBST) twice. The plates were incubated with test samples diluted in 2% skim milk in PBS for 2 h at room temperature. After being washed 3 times with PBST, the plates were incubated with an HRP-conjugated anti-mouse IgA antibody (Bethyl Laboratories) at room temperature for 1 h and then washed 3 times with PBST. Then 100 μl of the TMB solution (Thermofisher) was added to each well and incubated 15 min at room temperature. The reaction was stopped by addition of 3% H2504. Values at OD450 are shown.

FIG. 12B shows that QIV-vaccinated mice showed high levels of IgA antibody against the B/Bris virus noticeable at 5 dpc (compared to the PBS control challenge with B/Bris that shows no IgA responses). IgA responses are maintained in mice for at least 3 weeks after challenge. Virus specific IgA antibodies were detected by enzyme-linked immunosorbent assay (ELISA) using samples from lungs and nasal turbinates collected at 5 dpc and from feces, sera, nasal washes and BALF collected at 21 dpc. ELISA plates were coated with B/Bris overnight at 4° C. The plates were blocked with 10% skim milk in PBS for 2 h at room temperature and then washed with PBS containing 0.05% Tween 20 (PBST) twice. The plates were incubated with test samples diluted in 2% skim milk in PBS for 2 h at room temperature. After being washed 3 times with PBST, the plates were incubated with an HRP-conjugated anti-mouse IgA antibody (Bethyl Laboratories) at room temperature for 1 h and then washed 3 times with PBST. Then 100 μl of the TMB solution (Thermofisher) was added to each well and incubated 15 min at room temperature. The reaction was stopped by addition of 3% H2SO4. Values at OD450 are shown.

FIG. 12C shows that QIV-vaccinated mice showed high levels of IgG antibody against the ma Ca/04 (H1N1) virus noticeable at 5 dpc (compared to the PBS control challenge with ma Ca/04 (H1N1) that shows no IgG responses). IgG responses are maintained in mice for, at least, 3 weeks after challenge. Virus specific IgG antibodies were detected by enzyme-linked immunosorbent assay (ELISA) using samples from lungs and nasal turbinates collected at 5 dpc and from feces, sera, nasal washes and BALF collected at 21 dpc. ELISA plates were coated with ma-Ca/04 overnight at 4° C. The plates were blocked with 10% skim milk in PBS for 2 h at room temperature and then washed with PBS containing 0.05% Tween 20 (PBST) twice. The plates were incubated with test samples diluted in 2% skim milk in PBS for 2 h at room temperature. After being washed 3 times with PBST, the plates were incubated with an HRP-conjugated anti-mouse IgG antibody (Bethyl Laboratories) at room temperature for 1 h and then washed 3 times with PBST. Then 100 μl of the TMB solution (Thermofisher) was added to each well and incubated 15 min at room temperature. The reaction was stopped with 3% H2SO4 and OD450 values were read.

FIG. 12D shows that QIV-vaccinated mice showed high levels of IgG antibody against the B/Bris virus noticeable at 5 dpc (compared to the PBS control challenge with B/Bris that shows no IgG responses). IgG responses are maintained in mice for, at least, 3 weeks after challenge. Virus specific IgG antibodies were detected by enzyme-linked immunosorbent assay (ELISA) using samples from lung and nasal turbinate collected at 5 dpc and from feces, serum, nasal wash and BALF collected at 21 dpc. ELISA plates were coated with B/Bris overnight at 4° C. The plates were blocked with 10% skim milk in PBS for 2 h at room temperature and then washed with PBS containing 0.05% Tween 20 (PBST) twice. The plates were incubated with test samples diluted in 2% skim milk in PBS for 2 h at room temperature. After being washed 3 times with PBST, the plates were incubated with an HRP-conjugated anti-mouse IgG antibody (Bethyl Laboratories) at room temperature for 1 h and then washed 3 times with PBST. Then 100 μl of the TMB solution (Thermofisher) was added to each well and incubated 15 min at room temperature. The reaction was stopped with 3% H2SO4 and OD450 values were read.

FIG. 12E shows that QIV-vaccinated mice showed high levels of IgA and IgG antibody responses against the Ty/04 (H3N2) virus in nasal washes, BALF, and sera collected at 21 dpc with the heterologous ma Ca/04 (H1N1) or B/Bris viruses. Since HI responses did not change against the Ty04 (H3N2) virus after heterologous challenge (FIG. 11), it is reasonable to conclude that Ty04 (H3N2)-specific IgA and IgG responses were elicited by the QIV formulation. Anti-H3N2 IgA and IgG responses were maintained in mice for at least 3 weeks after challenge. At 21 dpc, IgA responses against B/Wisc were not different between the QIV group challenged with the heterologous ma Ca/04 (H1N1) virus and the PBS/non challenge control group. However, IgA responses against the B/Wisc virus were stimulated after challenge with the heterologous B/Bris virus, most likely due to some levels of cross-reactivity. Sera collected at 21 dpc suggest that the QIV formulation was capable of eliciting B/Bris-specific IgA and IgG responses, which were boosted during challenge with the heterologous B/Bris virus. Virus specific IgA and IgG antibodies were detected by ELISA as described in previous FIG. 12 panels using ELISA plates coated with Ty04 or B/Wisc virus. OD450 values are shown.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular Influenza B virus PB1 segment is disclosed and discussed and a number of modifications that can be made to a number of molecules including the Influenza B virus PB1 segment are discussed, specifically contemplated is each and every combination and permutation of Influenza B virus PB1 segment and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

In the background of IAVs, either avian- or swine-origin, incorporation of the single PB2 mutation (N265S) and the three PB1 mutations (K391E, D581G and A661T) from the MDV-A strain (most of these viruses naturally carry the NP D34G mutation) results in viruses with a ts phenotype in vitro. However, adequate att phenotype in vivo was only achieved after the incorporation of a C-terminal epitope tag in PB1 consisting of a 9-amino acid sequence derived from the H3 HA gene segment (HA tag) in the context of the ts mutations. Further studies showed that the IAV att viruses modified as described above (ts mutations plus HA-tag) are safe and efficacious as LAIVs in mice, chickens, and swine; and amenable for intranasal administration. In chickens, the IAV att vaccines were also suitable for immunization in ovo.

Despite some conserved features in virus structure, genome organization, and regulation of virus replication and transcription, IBV and IAV also exhibit a number of important distinctive characteristics, particularly on host range, virus prevalence, and evolutionary dynamics Herein, it was explored whether the ts mutations plus HA-tag in the IAV att strains would result in an att phenotype in the context of a prototypic IBV strain. Analogous mutations to those found in an IAV att alternative live virus vaccine were introduced into a prototypic B/Vic lineage strain, B/Brisbane/60/2008 (B/Bris). Specifically, mutations were engineered in PB2 K267S and PB1 (K391E, E580G and S660A). In addition, the PB1 segment was modified with the C-terminal HA tag in the presence or absence of temperature sensitive mutations. Attempts to stably maintain the PB2 K267S and PB1 K391E mutations in B/Bris failed. However, stability studies showed that the mutations E580G, and S660A and the C-terminal HA tag were stably maintained over 15 passages in SPF eggs and 20 passages in tissue culture cells. Safety and vaccination studies showed that the B/Bris strain with the PB1 segment carrying the E580G and S660A mutations and HA tag—herein referred to as B/Bris att—was stable, attenuated in vivo, and immunogenic. In mouse studies, intranasal immunization with B/Bris att resulted in sterilizing immunity against homologous challenge (B/Bris) and complete protection was achieved following heterologous challenge with a B/Yam lineage strain, B/Wisconsin/01/2010 (B/Wis). These studies show an alternative LAIV platform for development of IBV vaccines.

Accordingly, in one aspect, disclosed herein are live attenuated influenza B viruses comprising a substitution at residue 580 and/or residue 660 of the PB1 segment of the viral polymerase. In one aspect, the substitution of the glutamate at residue 580 and/or the serine at residue 660 of the PB1 segment of the viral polymerase can be a substitution with a nonpolar amino acid. For example, the substitution can be for a glycine, alanine, valine, leucine, or isoleucine. In one aspect, the substitution of the PB1 segment of the viral polymerase can be an E580G and/or a S660A substitution. For example, disclosed herein are live attenuated Influenza B viruses comprising an E580G mutation in the PB1 segment of the influenza viral polymerase, a S660A mutation in the PB1 segment of the influenza viral polymerase, or both an E580G mutation and a S660A mutation in the PB1 segment of the influenza viral polymerase.

It is understood and herein contemplated that the disclosed live attenuated influenza B virus can be derived from any influenza B virus known. In one aspect, the live attenuated B virus can be an Influenza B virus of the Victoria lineage (for example, Influenza B/Brisbane, Influenza B/Malaysia) or an Influenza B virus of the Yamagata lineage (for example, Influenza B/Florida, Influenza B/Phuket, Influenza B/Shanghai, Influenza B/Massachusetts, or Influenza B/Wisconsin).

In one aspect, the live attenuated influenza B viruses can further comprise a HA-tag. Such a tag can be located in-frame at the C-terminus of PB1. In one aspect, the HA tag can comprise the sequence YPYDVPDY (SEQ ID NO: 2).

Also disclosed herein are vaccines comprising any of the live attenuated Influenza B viruses disclosed herein or synthetic viruses comprising segments of the modified live attenuated influenza B viruses disclosed herein. For example, disclosed herein are vaccines comprising a live attenuated influenza B virus wherein the PB1 segment of the Influenza B virus polymerase comprising an E580G substitution, a S660A substitution, and/or a HA-tag. In one aspect, disclosed herein are live attenuated influenza B viruses or vaccines comprising a live attenuated influenza B virus wherein the PB1 segment of the Influenza B virus polymerase comprising an E580G substitution and/or a S660A substitution, and a HA-tag.

The vaccines comprising live attenuated Influenza B viruses can be univalent or multivalent (i.e., bivlent, trivalent, quadrivalent, or pentavalent) vaccine directed only to influenza B viruses or comprise additional valency for influenza A viruses. For example, the live attenuated vaccine can comprise one or more live attenuated Influenza B virus of the Victoria lineage (for example, Influenza B/Brisbane, Influenza B/Malaysia) and/or one or more live attenuated Influenza B virus of the Yamagata lineage (for example, Influenza B/Florida, Influenza B/Phuket, Influenza B/Shanghai, Influenza B/Massachusetts, or Influenza B/Wisconsin), wherein at least one of the live attenuated influenza B strains comprises a substitution at residue 580 and/or 660 of the PB1 segment of the viral polymerase as disclosed herein. Also, vaccines comprising said viruses can comprise valency to Influenza A and B. Thus, disclosed herein are vaccine comprising one or more live attenuated influenza A strains and one or more live attenuated influenza B strains, wherein at least one influenza B strain comprises a substitution at residue 580 and/or 660 of the PB1 segment of the viral polymerase (for example, the vaccine can be a quadrivalent live attenuated influenza virus vaccine comprising two live attenuated Influenza A virus strains (a live attenuated H3N3 virus and a live attenuated H1N1 virus) and one or more live attenuated Influenza B virus; wherein at least one of the live attenuated Influenza B viruses comprises a substitution at residue 580 and/or 660 of the PB1 segment of the viral polymerase (for example, a E580G and/or S660A substitution); and wherein one or more of the live attenuated Influenza B viruses is of the Victoria lineage (for example, Influenza B/Brisbane, Influenza B/Malaysia) and/or one or more live attenuated Influenza B virus is of the Yamagata lineage (for example, Influenza B/Florida, Influenza B/Phuket, Influenza B/Shanghai, Influenza B/Massachusetts, or Influenza B/Wisconsin). For example, disclosed herein are multivalent vaccines (for example, a quadrivalent vaccine) comprising a live attenuated H3N3 virus, a live attenuated H1N1 virus, a live attenuated Influenza B/Brisbane virus, and a live attenuated Influenza B/Wisconsin virus, wherein at least one influenza B strain comprises a substitution at residue 580 and/or 660 of the PB1 segment of the viral polymerase (for example, a E580G and/or S660A substitution).

### 1. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

Following administration of a disclosed live attenuated Influenza B viruses or vaccine comprising said live attenuated influenza B virus, for treating, inhibiting, or preventing an Influenza B infection, the efficacy of the attenuated virus can be assessed in various ways well known to the skilled practitioner. For instance, one of ordinary skill in the art will understand that a composition, such as the live attenuated Influenza B virus, disclosed herein is efficacious in treating or inhibiting an Influenza B infection in a subject by observing that the composition reduces viral load.

The compositions that inhibit Influenza B infections disclosed herein may be administered prophylactically to patients or subjects who are at risk for being exposed to Influenza B virus or have been newly exposed to Influenza B virus.

### C. Methods of Inhibiting and/or Preventing an Influenza B Virus Infection

It is understood and herein contemplated the live attenuated Influenza B viruses disclosed herein and vaccines comprising said viruses can be used to immunize a subject against exposure and infection to an Influenza virus, for example, an Influenza B virus. In one aspect, disclosed herein are methods of inhibiting and/or preventing an Influenza B virus infection comprising administering to a subject a live attenuated influenza B viruses comprising a substitution at residue 580 and/or 660 of the PB1 segment of the viral polymerase. In one aspect, the substitution of the PB1 segment of the viral polymerase can be a substitution of the glutamate at residue 580 and/or the serine at residue 660 of the PB1 segment of the viral polymerase with a nonpolar amino acid (i.e., glycine, alanine, valine, leucine, or isoleucine), such as, an E580G and/or a S660A substitution. For example, disclosed herein are methods of inhibiting and/or preventing an Influenza B virus infection comprising administering to a subject a live attenuated Influenza B viruses comprising an E580G and S660A mutations in the PB1 segment of the influenza viral polymerase.

It is understood and herein contemplated that the live attenuated Influenza B viruses for use in the disclosed methods can be derived from any Influenza B virus, including any Yamagata or Victoria lineage Influenza B virus, including but not limited to live attenuated Influenza B virus of the Victoria lineage (for example, Influenza B/Brisbane, Influenza B/Malaysia) and/or live attenuated Influenza B viruses of the Yamagata lineage (for example, Influenza B/Florida, Influenza B/Phuket, Influenza B/Shanghai, Influenza B/Massachusetts, or Influenza B/Wisconsin).

In one aspect, the live attenuated influenza B viruses used in the disclosed methods can further comprise a HA-tag. Such a tag can be located in-frame at the C-terminus of PB1. In one aspect, the HA tag can comprise the sequence YPYDVPDY (SEQ ID NO: 2). Thus, in one aspect, disclosed herein are methods of inhibiting and/or preventing an Influenza B virus infection comprising administering to a subject a live attenuated influenza B virus wherein the PB1 segment of the Influenza B virus polymerase comprising an E580G substitution, a S660A substitution, and/or a HA-tag.

In one aspect, the live attenuated Influenza B viruses in the disclosed methods for inhibiting and/or preventing influenza B infections can be a component of a composition such as a vaccine. Thus, disclosed herein are methods of inhibiting and/or preventing an Influenza B virus infection comprising administering to a subject a vaccine comprising one or more live attenuated Influenza B viruses, wherein the live attenuated Influenza B virus comprises a substitution of the glutamate at residue 580 and/or the serine at residue 660 (for example, a E580G and S660A substitution) in the PB1 segment of the influenza viral polymerase. Also disclosed herein are methods of inhibiting and/or preventing an Influenza B virus infection comprising administering to a subject a vaccine comprising a live attenuated influenza B virus wherein the PB1 segment of the Influenza B virus polymerase comprising an E580G substitution, a S660A substitution, and a HA-tag.

In one aspect, the live attenuated B virus or viruses administered to a subject for inhibiting an Influenza B virus infection can be a component in a multivalent vaccine (such as a bivalent, trivalent, or quadrivalent vaccine) directed to inhibiting Influenza B viruses. For example, the live attenuated vaccine used in the disclosed methods of Inhibiting Influenza B virus can comprise administering to the subject one or more live attenuated Influenza B virus of the Victoria lineage (for example, Influenza B/Brisbane, Influenza B/Malaysia) and/or one or more live attenuated Influenza B virus of the Yamagata lineage (for example, Influenza B/Florida, Influenza B/Phuket, Influenza B/Shanghai, Influenza B/Massachusetts, or Influenza B/Wisconsin), wherein at least one of the live attenuated influenza B strains comprises a substitution at residue 580 and/or 660 of the PB1 segment of the viral polymerase as disclosed herein (for example, a E580G and S660A substitution). In one aspect, the multivalent vaccine used in the disclosed methods can comprise 2, 3, 4, or 5 Victoria lineage influenza B viruses, 2, 3, 4, or 5 Yamagata lineage influenza B virus, or any combination of 2, 3, 4, or 5 Victoria and Yamagata lineage viruses provided at least one influenza B virus is a live attenuated influenza B virus comprising a substitution of the glutamate at residue 580 and/or the serine at residue 660 (for example, a E580G and S660A substitution) in the PB1 segment of the influenza viral polymerase.

It is also understood and herein contemplated the live attenuated Influenza B viruses disclosed herein and vaccines comprising said viruses can be used to immunize a subject against exposure and infection to any influenza viral infection including both Influenza A and Influenza B infections as part of a multivalent pan-influenza vaccine (for example, a bivalent, trivalent, quadrivalent, or pentavalent vaccine). In one aspect, disclosed herein are methods of inhibiting and/or preventing an Influenza virus infection (including, Influenza A (such as, for example, H3N2 and H1N1) and Influenza B) comprising administering to a subject a live attenuated influenza B viruses comprising a substitution at residue 580 and/or 660 of the PB1 segment of the viral polymerase, wherein the live attenuated influenza B virus is a component in a multivalent live attenuated influenza vaccine. In one aspect, the multivalent live influenza vaccine used in the disclosed methods can comprise one or more live attenuated influenza A strains and one or more live attenuated influenza B strains, wherein at least one influenza B strain comprises a substitution at residue 580 and/or 660 of the PB1 segment of the viral polymerase (for example, the vaccine can be a quadrivalent live attenuated influenza virus vaccine comprising two live attenuated Influenza A virus strains (a live attenuated H3N3 virus and a live attenuated H1N1 virus) and one or more live attenuated Influenza B virus of the Victoria lineage (for example, Influenza B/Brisbane, Influenza B/Malaysia) and/or one or more live attenuated Influenza B virus of the Yamagata lineage (for example, Influenza B/Florida, Influenza B/Phuket, Influenza B/Shanghai, Influenza B/Massachusetts, or Influenza B/Wisconsin). Thus, for example, disclosed herein are methods of inhibiting and/or preventing an influenza infection comprising administering to a subject a multivalent vaccine (for example, a quadrivalent vaccine) comprising a live attenuated H3N3 virus, a live attenuated H1N1 virus, a live attenuated Influenza B/Brisbane virus, and a live attenuated Influenza B/Wisconsin virus, wherein at least one influenza B strain comprises a substitution at residue 580 and/or 660 of the PB1 segment of the viral polymerase (for example, a E580G and/or S660A substitution).

### D. Methods of Attenuating an Influenza B Virus

As noted herein, substitution at residue 580 and/or 660 of the PB1 segment of the viral polymerase of an Influenza B virus will attenuate the virus. Accordingly, in one aspect, disclosed herein are methods of attenuating an Influenza B virus (including, but not limited to any Victoria lineage (for example, Influenza B/Brisbane, Influenza B/Malaysia) and/or Yamagata lineage (for example, Influenza B/Florida, Influenza B/Phuket, Influenza B/Shanghai, Influenza B/Massachusetts, or Influenza B/Wisconsin) virus disclosed herein) comprising substituting the glutamate at residue 580 and/or the serine at residue 660 of the PB1 segment of the viral polymerase with a nonpolar amino acid. For example, the substitution can be for a glycine, alanine, valine, leucine, or isoleucine. In one aspect, disclosed herein are methods of attenuating an Influenza B virus comprising substituting the glutamate at residue 580 and/or the serine at residue 660 of the PB1 segment of the viral polymerase with a nonpolar amino acid, wherein the substitution of the PB1 segment of the viral polymerase comprises an E580G and/or a S660A substitution.

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1 Live Attenuated Influenza B Virus

### a) Materials and Methods

### (1) Cells Lines and Virus Strains

Madin-Darby canine kidney (MDCK) and human embryonic kidney 293T cells were maintained in Dulbecco's modified Eagle's medium (DMEM) supplemented with 10% fetal bovine serum (FBS) and 1% antibiotics/antimycotic solution (Sigma-Aldrich, St. Louis, Mo.). Cells were propagated at 37° C. in a humidified incubator under 5% CO₂ atmosphere. B/Brisbane/60/2008 (WT B/Bris) and B/Wisconsin/01/2010 (WT B/Wis) influenza B virus strains were a gift from Ruben Donis, the Centers for Diseases Control and Prevention, Atlanta, Ga. Virus stocks were amplified in specific pathogen free embryonated chicken eggs (SPF eggs) (B&E Eggs, York Springs, Pa. or Charles River, Norwich, Conn.) and stored at −80° C. Viruses used in this report are summarized in Table 1.

TABLE 1
Influenza viruses used in the present study

| Virus | Designated abbreviation |
|---|---|
| B/Brisbane/60/2008^{a} | WT B/Bris |
| Reverse-genetics B/Brisbane/60/2008^{b} | WT RG-B/Bris |
| B/Wisconsin/01/2010^{c} | WT B/Wis |
| B/Brisbane/60/2008 ts^{d} | B/Bris ts |
| B/Brisbane/60/2008 att^{e} | B/Bris att |
| B/Brisbane/60/2008 PB2 W359F^{f} | B/Bris PB2-W359F |
| B/Brisbane/60/2008 PB2 F406Y^{g} | B/Wis PB2-F406Y |
| 2 B/Wisconsin/01/2010:6 B/Bris PB2 F406Y^{h} | B/Wis PB2-F406Y |
| 1 A/mallard/Alberta/24/01 (H7N3):7 | 7attWF10:1malH7 |
| A/guinea fowl/Hong Kong/WF10/1999 | |
| (H9N2)ⁱ | |

^{a}Wild type B/Brisbane/60/2008 virus.
^{b}Wild type B/Brisbane/60/2008 virus generated by reverse genetics.
^{c}Wild type B/Wisconsin/01/2010 virus.
^{d}B/Brisbane/60/2008 virus carrying temperature sensitive mutations (E580G and S660A) in PB1.
^{e}B/Brisbane/60/2008 virus carrying temperature sensitive mutations (E580G and S660A) and HA tag in PB1.
^{f}B/Brisbane/60/2008 virus carrying W359F mutation in PB2.
^{g}B/Brisbane/60/2008 virus carrying F406Y mutation in PB2.
^{h}Reassortant virus containing HA and NA genes of B/Wisconsin/01/2010 and the same internal genes constellation of B/Brisbane/60/2008 F406Y virus.
ⁱReassortant virus containing HA of A/mallard/Alberta/24/01 (H7N3) and 7 remaining genes of the attenuated A/guinea fowl/Hong Kong/WF10/1999 (H9N2) virus (WF10att).

### (2) Animal Use and Compliance

Five- to six-week old female DBA/2J mice (Charles River, Frederick, Md. or The Jackson Laboratory, Bar Harbor, Me.) were used in all mouse experiments. All animal studies were performed under animal biosafety level 2 (ABSL-2) containment conditions following protocols approved by the respective Institutional Animal Use and Care Committees (IACUC) at the University of Georgia and University of Maryland, College Park. Mice that experienced significant weight loss (21-25%) or scored three or higher on a four-point scale of disease severity were humanely euthanized.

### (3) Cloning and Site Directed Mutagenesis

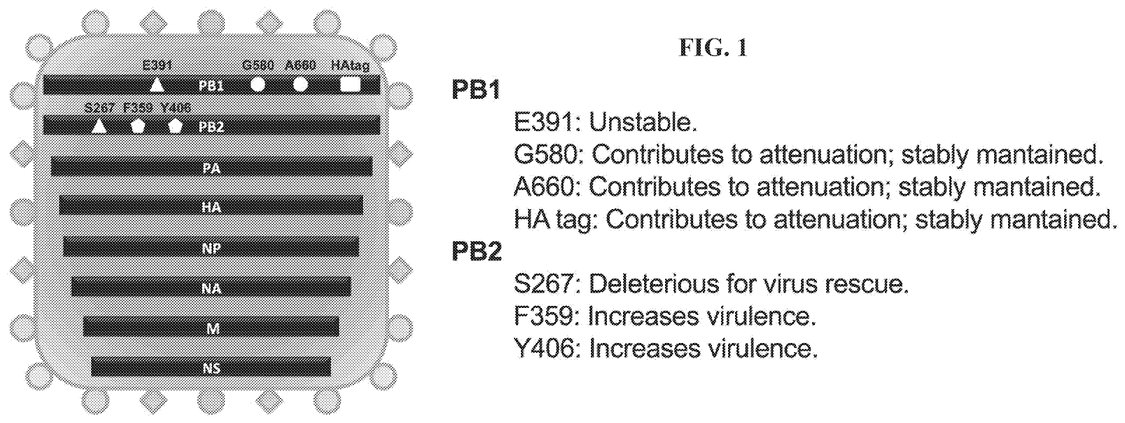

The reverse genetics system for WT B/Bris strain has been described elsewhere. HA and NA surface gene segments of WT B/Wis were cloned into the bi-directional cloning vector pDP2002 by standard cloning techniques. To generate the attenuated PB1 segment (PB1 att), a modified HA tag was first cloned in-frame at the C-terminus of PB1 by inverse PCR. The cloning strategy was based after the HA tag cloning procedure with minor modifications. Primers were designed such that the original stop codon in PB1 was mutated to an alanine (A) and a new one was introduced following the modified HA tag. Additionally, the codon for the last amino acid present in the wild type PB1 sequence (Isoleucine, I) was repeated immediately before the introduced alanine (A) codon. Thus, the entire amino acid sequence introduced into PB1 was IAYPYDVPDY (SEQ ID NO: 1), with the final 8 amino acids (italics, underscored) corresponding to the modified HA tag. Subsequently, the mutations K391E, E580G, and S660A were introduced into PB1 via site-directed mutagenesis using PCR. The mutations K267S, F406Y, or W359F were introduced into the PB2 segment of the B/Brisbane/60/2008 genome by site-directed mutagenesis with the QuickChange II XL kit (Agilent, Santa Clara, Calif.). PCR reactions were performed with either Pfu Ultra DNA Polymerase AD (Agilent, Santa Clara, Calif.) or Phusion High Fidelity DNA polymerase (New England Biolabs, Ipswich, Mass.). All constructed plasmid were sequenced and no unwanted mutations were identified. A schematic overview of all the modifications introduced into the IBV genome and their respective outcomes is depicted in FIG. 1.

### (4) Generation of Recombinant Viruses by Reverse Genetics

Viruses were rescued using a co-culture of 293T/MDCK cells. To generate B/Bris att, B/Bris PB2-F406Y, and B/Bris PB2-W359F viruses, the respective mutant plasmid was paired with the 7 remaining WT B/Bris plasmids. B/Wis PB2-F406Y virus is a 2:6 reassortant carrying the surface genes of WT B/Wis and the internal gene constellation of B/Bris PB2-F406Y virus. Following transfection, transfected cells were incubated at 35° C. After 24 h incubation, media was replaced with Opti-MEM I (Life Technologies, Carlsbad, Calif.) containing 1 μg/mL TPCK-trypsin (Worthington Biochemicals, Lakewood, N.J.) and antibiotic/antimycotic solution. Following virus rescue (Passage 0, P0), the tissue culture supernatant was used to inoculate fresh MDCK cells to amplify the virus once (P1). Viruses in P1 were used for further amplify in MDCK cells (P2) and then in SPF eggs (E1). The viruses obtained in E1 were used to grow virus stocks in SPF eggs (E2). P1, P2, E1 and E2 virus amplifications were carried out at 33° C. Virus stocks were titrated by both tissue culture infectious dose 50 (TCID₅₀) and egg infectious dose 50 (EID₅₀). Virus titers were determined by the Reed and Muench method.

### (5) Stability of Attenuated Influenza B Virus

All the virus stocks were sequenced by either Sanger and/or next-generation sequencing (NGS) to assess the presence of the inserted mutations in rescued viruses. To ascertain stability of attenuation markers, the B/Bris att virus (E2) was serially passaged either up to 20 times in MDCK cells or up to 15 times in SPF eggs. RNA from tissue culture supernatant and allantoic fluid, respectively, was purified using the RNeasy mini kit (Qiagen, Valencia, Calif.). Isolated RNA served as template for a one-step RT-PCR whole IBV genome amplification and NGS using Illumina MiSeq platform. Additionally, pooled nasal turbinate homogenates obtained from mice at 3 and 5 days post-vaccination (dpv) were used for RNA isolation. The PB1 segment was amplified using a two-step RT-PCR reaction and sequenced to verify the stability of the B/Bris att virus following immunization.

### (6) Mini-Genome Assays

To assess viral polymerase activity, a Gaussia Luciferase (GLuc) reporter plasmid (pBNPGLuc) was constructed. The open reading frame (ORF) of GLuc was amplified by PCR from pGLuc-Basic (New England Biolabs, Ipswich, Mass.) and subcloned into pDP2002. The plasmid was engineered such that GLuc ORF was flanked by the 5′ and 3′ untranslated regions (UTRs) of the IBV NP segment. This construct was further modified by removing the pCMV promoter immediately upstream of 5′-UTR. The resulting plasmid was verified by sequencing that revealed no unwanted modifications. The vRNP minigenome assay was reconstituted in 293T cells co-transfected with pCMV-SEAP, pBNPGLuc, and B/Bris PB2, PB1, PA and NP plasmids using TransIT-LT1 transfection reagent (Mirus, Madison, Wis.) following manufacturer's instructions. The pCMV-SEAP plasmid expresses the secreted alkaline phosphatase and it was used to normalize for transfection efficiency. At indicated time points, tissue culture supernatant from transfected cells was collected to measure reporter activity using a Victor ×3 Multilabel Plate Reader (PerkinElmer, Waltham, Mass.). GLuc activity was assessed using the Biolux Gaussia Luciferase Assay Kit (New England Biolabs, Ipswich, Mass.) while phosphatase activity was measured using the Phospha-light SEAP Reporter Gene Assay System (Life Technologies, Carlsbad, Calif.). Experiments were carried out independently at least twice and all transfection conditions were tested in triplicate per experiment.

### (7) Western Blotting

Confluent MDCK cells were inoculated with influenza viruses (WT RG-B/Bris, B/Bris ts, B/Bris att or 7attWF10:1malH7) at 1 MOI for 1 h at 37° C. The 7attWF10:1malH7 virus is a reassortant IAV att control virus. Following inoculation, infected cells were incubated at 33° C. for 20 h. The supernatant was then removed and cells lysed with 150 μL of Laemmli Buffer containing β-mercaptoethanol (Bio-Rad, Berkeley, Calif.). Cell lysates were boiled for 7 min followed by brief sonication. Proteins were separated on a 4-20% sodium dodecyl sulfate-polyacrylamide gel electrophoresis (SDS-PAGE) and transferred onto nitrocellulose membranes (Bio-Rad, Berkeley, Calif.) for immunoblot analysis. The membranes were blocked in 5% molecular grade non-fat dry milk (NFDM, Bio-Rad, Berkeley, Calif.) for 2 h at room temperature, followed by incubation with an anti-GAPDH mouse primary antibody (Santa Cruz Biotech, Dallas, Tex.) for 2 h at room temperature or a mouse anti-HA-tag primary antibody (Cell Signaling Technologies, Danvers, Mass.) overnight at 4° C. After washing, the membranes were incubated with a goat anti-mouse IgG antibody conjugated to horseradish peroxidase (Southern Biotech, Birmingham, Ala.) Immunoreactive proteins were visualized by autoradiography using an enhanced chemiluminescence reagent (Clarity Western ECL Substrate, Bio-Rad, Berkeley, Calif.).

### (8) In Vitro Growth Kinetics

Growth kinetics of the WT RG-B/Bris and B/Bris att viruses were compared at different temperatures. Confluent monolayers of MDCK cells were inoculated at 0.01 MOI of each virus. At indicated time points, tissue culture supernatant from inoculated cells was collected for virus titer quantification. Virus titers were determined by TCID₅₀ using the Reed and Muench method. Experiments were carried out independently at least twice and all conditions were tested in triplicate per experiment.

### (9) Safety Assessment of B/Bris PB2 Mutant Viruses

To evaluate the impact of the PB2 F406Y and W359F mutations on increased IBV virulence, DBA/2J mice (Charles River, Frederick, Md.) were randomly distributed into 4 groups (n=10 per group). Each mouse was anesthetized with isoflurane and subsequently inoculated via the intranasal (I.N.) route. Groups 1 received 50 μL of phosphate-buffered saline (PBS) and served as a control group. Each mouse in groups 2, 3 and 4 was inoculated with 10⁵ EID₅₀ in 50 μL of PBS of the B/Bris PB2-F406Y, B/Bris PB2-W359F or WT RG-B/Bris viruses, respectively. Clinical signs of disease, body weight changes and mortality were monitored daily.

### (10) Vaccination and Challenge Studies

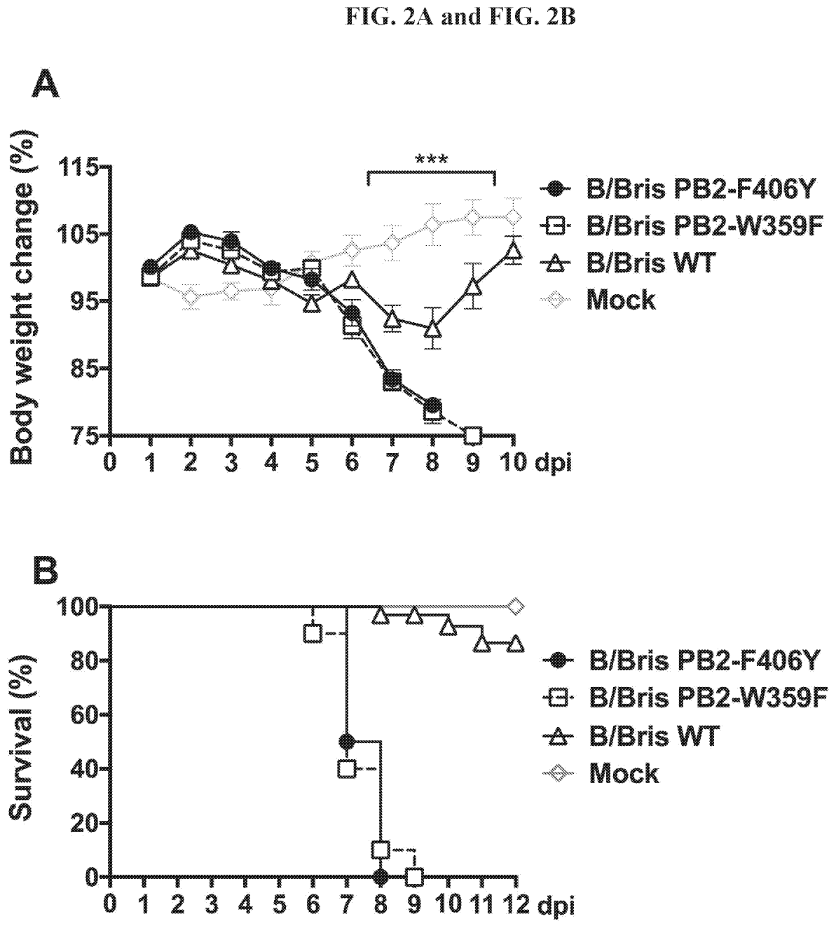

To evaluate the immune response and protective efficacy of B/Bris att, DBA/2J mice (Charles River, Frederick, Md. or The Jackson Laboratory, Bar Harbor, Me.) were randomly distributed into 5 groups (n=24 per group). Each mouse was anesthetized with isoflurane and subsequently inoculated I.N. Groups 1, 2, and 3 received 50 μL of PBS alone as mock-vaccinated control groups. Groups 4 and 5 were inoculated with 10⁶ EID₅₀ in 50 μL of PBS of the B/Bris att virus per mouse. An additional group (n=8) received 10⁶ EID₅₀ per mouse of the WT RG-B/Bris virus and served as control to evaluate in vivo the attenuated phenotype of B/Bris att. At 3 and 5 days post-inoculation (dpi), 3 or 4 animals from each group were humanely euthanized for tissue collection. Nasal turbinates and lungs were collected for virus titer quantification and histopathological examination. The day before challenge (20 dpi), mice were bled from the submandibular vein to measure neutralizing antibody responses. At 21 dpi, Group 1 remained unchallenged while Groups 2, 3, 4 and 5 (n=16 per group) were challenged I.N. with 10⁷ EID₅₀ per mouse in 50 μL of PBS of either B/Bris PB2-F406Y or B/Wis PB2-F406Y viruses. IBV natural isolates are usually not lethal to mice unless the virus is serially adapted in this species or a high virus dose is used for inoculation. The increased virulence observed in mice inoculated with the B/Bris PB2-F406Y and B/Bris PB2-W359F viruses (FIGS. 2A and B) prompted the selection of the B/Bris PB2-F406Y virus for lethal challenge in vaccine efficacy studies. Similarly, the B/Wis PB2-F406Y virus was created for heterologous (antigenically distinct) challenge. At 3 and 5 days post-challenge (dpc), 4 mice per group were humanely euthanized for tissue collection as described above. At 21 dpc, all remaining mice (n=8/group) were bled from the submandibular vein for serology. Clinical signs of disease, body weight changes and mortality were monitored daily throughout the study.

### (11) HI Assay

Serum samples collected at 20 dpi and at 21 dpc were assayed for the presence of neutralizing antibodies by HI assay as described (WHO, 2002) using the WT B/Bris or WT B/Wis viruses as antigen.

### (12) Histopathology

At indicated time points, lungs from mice euthanized post-vaccination and post-challenge were collected and preserved in 10% formalin for histopathological examination by hematoxylin & eosin (H&E) staining. Tissue sections from bronchi/bronchioles, pulmonary vasculature, alveoli and the overall extent of pulmonary lesions were scored on a 0-4 point scale, based on the increased level of tissue damage and inflammation. Representative images of lung histopathology were taken at 20× magnification.

### (13) Statistical Analyses

All data analyses were performed using GraphPad Prism Software Version 7 (GraphPad Software Inc., San Diego, Calif.). All in vitro assays were performed a minimum of two times in triplicate. For multiple comparisons, two-way ANOVA was performed followed by a post-hoc Bonferroni test. Differences in survival curves were analyzed using the log-rank test. A P value below 0.05 (P<0.05) was considered significant.

### b) Results

### (1) Incorporation and Maintenance of Attenuation Markers in an Influenza B Virus

The amino acid substitutions associated with the is phenotype of the MDV-A strain have been mapped to the vRNP complex and shown to be transferable to other IAV backbones. Early work carried out by the laboratory demonstrated these substitutions alone do not confer adequate levels of attenuation in vivo (i.e. defined as either no or significant reduction in replication in the lower respiratory tract). The additional in-frame incorporation of a C-terminal tag in PB1 of IAV viruses (from either avian- or swine-origin) was required to achieve adequate attenuation in vivo.

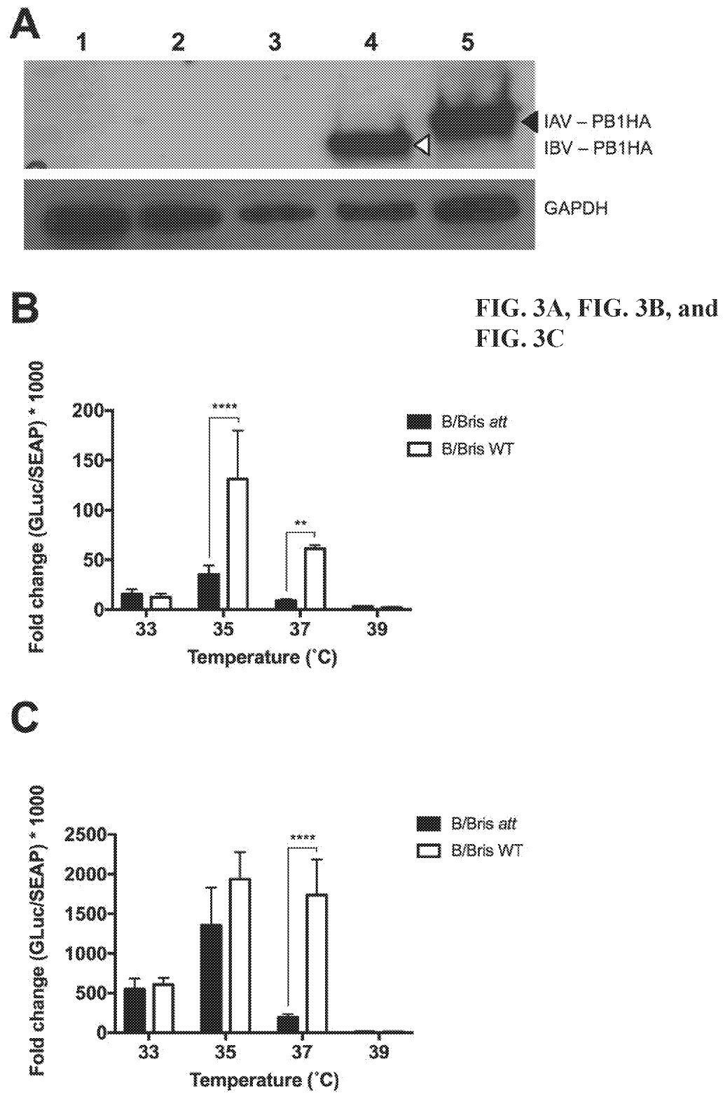

In this study, as an evaluation was conducted as to whether modifications of Influenza B virus and the resulting att phenotype could be developed (FIG. 1). Specifically, using the B/Brisbane/60/2008 strain, mutations in PB2 (K267S), PB1 (K391E, E580G, and S660A), and a chimeric PB1 carrying a C-terminal HA tag (in the presence or absence of ts mutations) were evaluated. Attempts to rescue an IBV virus carrying the PB2 K267S mutation resulted in instability at such position, therefore efforts were redirected to produce a virus with alternative PB2 mutations. IBV viruses carrying either the PB2 F406Y or PB2 W359F mutations were produced, as either one of those mutations affect the protein's cap-binding and the virus' polymerase activity. Surprisingly, the B/Bris PB2-F406Y and B/Bris PB2-W359F viruses showed increased virulence in DBA/2J mice (FIG. 2). Therefore, further modifications of the B/Bris genome were focused on the PB1 segment. B/Bris mutant viruses carrying the mutations K391E, E580G, and S660A were produced with or without the C-terminal HA tag (FIG. 3A). Independent virus rescue attempts revealed that the K391E mutation was readily detected in early virus passage in MDCK cells (P2) but quickly lost after passage in SPF eggs (E2). While the temperature sensitive phenotype of IAV backbones is primarily determined by the PB1 K391E and E581G mutations, the data presented below indicate that the K391E mutation is not critical for attenuation in the context of an IBV virus. Since the B/Bris mutant virus with the combined E580G, S660A, and HA tag modifications (herein referred to as B/Bris att) was stable over several passages in either tissue culture cells or SPF eggs, it was further characterized to establish whether it contained a ts phenotype in vitro and/or if it was attenuated in vivo.

### (2) B/Bris att Displays a Temperature Sensitive Phenotype

In vitro characterization of the B/Bris att vaccine candidate began by evaluating polymerase activity (measured by levels of GLuc expression) in vRNP minigenome assays (FIGS. 3B and C). The corresponding components of the IBV vRNP complex including PB2, PB1 (or its mutant), PA, NP, and the pBNPGLuc reporter plasmid, along with the pCMV-SEAP plasmid were co-transfected in 293T cells. GLuc reporter activity was normalized by SEAP enzyme activity and served as surrogate of virus polymerase complex activity. Following transfection, cells were maintained at 33°, 35°, 37° and 39° C. At the indicated time points, supernatant from transfected cells were harvested to measure reporter activity. The results showed that at 33° C., both B/Bris att and WT RG-B/Bris vRNP complexes displayed similar levels of GLuc reporter activity at either 24 (P=ns) or 48 (P=ns) h post-transfection (hpt) (FIGS. 3B and C, respectively). At 35° C. both vRNP complexes reached peak polymerase activity although the B/Bris att polymerase complex exhibited reduced luciferase activity compared to WT RG-B/Bris (P<0.0001 at 24 hpt). A temperature increase to 37° C. further accentuated statistically significant differences (P<0.001 at 24 h and P<0.0001 at 48 h) in levels of luciferase activity between the B/Bris att and WT RG-B/Bris polymerase complexes at either time points analyzed, indicating modifications in PB1 confer a ts phenotype to B/Bris att. As expected, at 39° C. both B/Bris att and WT RG-B/Bris polymerase complexes displayed residual levels of luciferase activity.

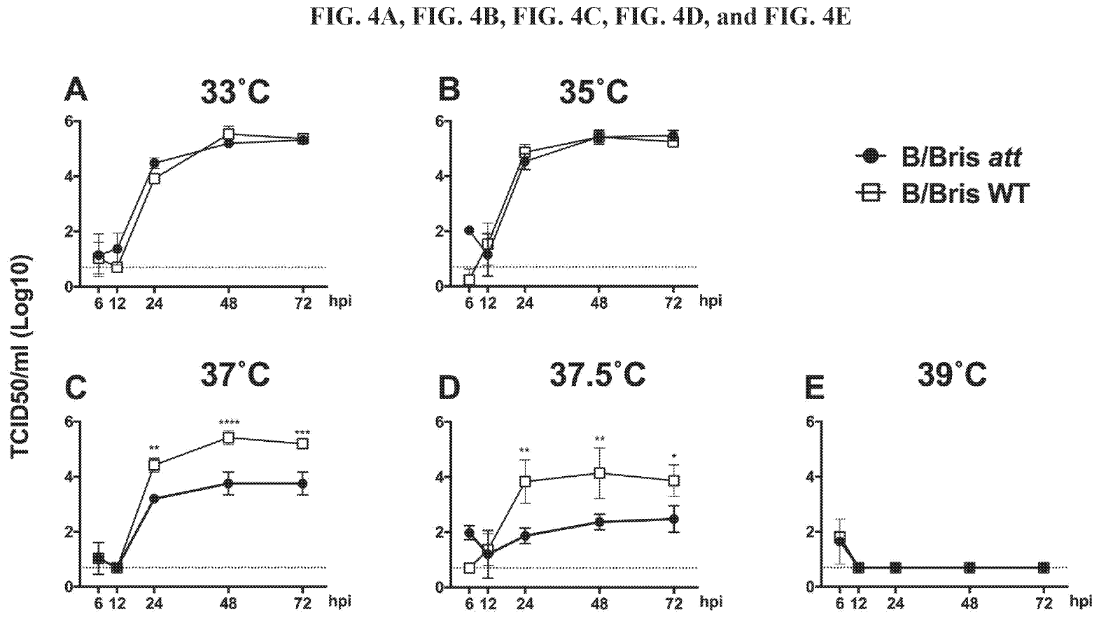

In order to further characterize the impact of PB1 modifications in the ts phenotype, growth kinetics of the B/Bris att and WT RG-B/Bris viruses were evaluated. Confluent monolayers of MDCK cells were infected at 0.01 MOI and virus growth kinetics were monitored at 33°, 35°, 37°, 37.5° and 39° C. Overall the growth kinetics results showed a similar trend as observed with the vRNP reconstitution assays. Both B/Bris att and WT RG-B/Bris viruses grew to similar titers at 33° C. (FIG. 4A) (P=ns regardless of the time point tested). In contrast to the virus polymerase complex assay results, the B/Bris att and WT RG-B/Bris viruses also grew to similar titers at 35° C. (FIG. 4B) (P=ns at 12, 24, 48 and 72 hpi). At 37° C., the WT RG-B/Bris virus grew to similar levels as those observed at 33° C. and 35° C. (FIG. 4C). In contrast, B/Bris att showed impaired virus growth at 37° C. at either 24 (P<0.01), 48 (P<0.0001) or 72 (P<0.001) h post-infection (hpi) compared to the WT RG-B/Bris virus (FIG. 4C). A half-degree increment in temperature led to further decrease in the B/Bris att virus growth reaching a ˜2-log reduction in virus titer at 24 hpi (P<0.01) when compared to WT RG-B/Bris (FIG. 4D). None of the viruses grew to detectable levels at 39° C., consistent with the vRNP reconstitution assay data (FIG. 4E). Taken together, these results indicate that modifications introduced in PB1 resulted in a B/Bris att virus with a ts phenotype in vitro.

### (3) B/Bris att Virus can be Safely Administrated in Mice and is Immunogenic

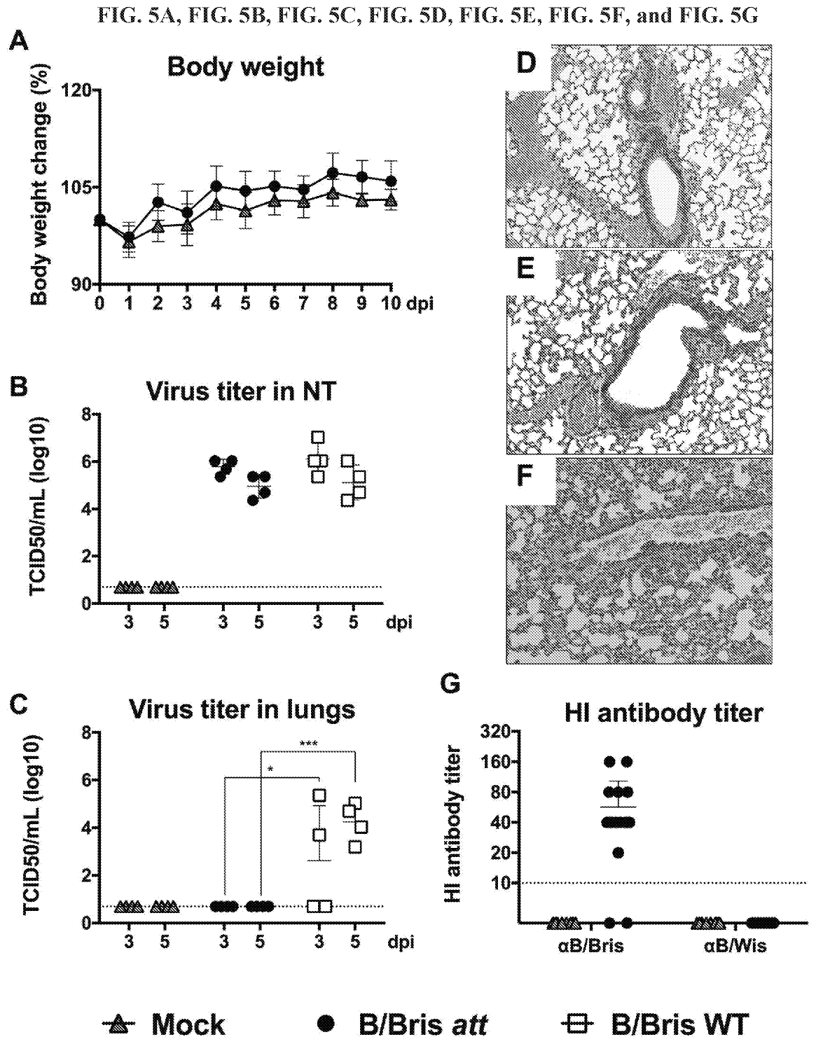

To investigate whether the ts phenotype observed in vitro would result in the B/Bris att virus attenuation in vivo, its safety was evaluated in mice. Groups of DBA/2J mice were randomly distributed in experimental groups. Each mouse were I.N. inoculated with either PBS (mock), 10⁶ EID₅₀ of the B/Bris att virus, or 10⁶ EID₅₀ of the WT RG-B/Bris virus. Clinical signs of disease, body weight changes and mortality were recorded daily. Body weight monitoring revealed no weight loss following inoculation of the B/Bris att vaccine candidate (FIG. 5A). A subset of mice (n=4/group/time point) was euthanized at 3 and 5 dpi in order to determine the levels of virus replication in nasal turbinates (NT) and lungs; the upper and lower respiratory tract, respectively. Both B/Bris att and WT RG-B/Bris viruses replicated well in the upper respiratory tract as evidenced by virus detection in nasal turbinates (P=ns, FIG. 5B). These results are in agreement with vRNP reconstitution assays and growth kinetics experiments performed at 33° C. In contrast, only mice inoculated with the WT RG-B/Bris virus had detectable levels of virus replication in the lower respiratory tract as shown by significant virus levels in the lungs (FIG. 5C) at 3 (P<0.05) and 5 dpi (P=0.001). These results are further supported by histopathology findings of lung samples collected at 5 dpi. Lungs were scored on a 0-4 point scale where a score of four (4) indicates severe lung pathology. Lungs of mice either mock-inoculated or inoculated with the B/Bris att virus had an average whole lung score of zero (0) at 5 dpi (Table 2, FIGS. 5D and 5E). In contrast, lungs of mice inoculated with the WT RG-B/Bris virus received an average whole lung score of two (2) at 5 dpi, characterized by the presence of mild lesions in the lungs and debris in the bronchial lumen (Table 2, FIG. 5F). These findings are consistent with the vRNP reconstitution assays and growth kinetics experiments, which showed reduced polymerase activity and virus growth at 37° C. for B/Bris att.

TABLE 2
Histopathological findings of mouse lungs

| | | |
|---|---|---|
| Group | Whole lung grade | Major findings |

Post inoculation (5 dpi)

| | | |
|---|---|---|
| Control | − | — |
| B/Bris att | − | Presence of macrophages, |
| | | minimal alveolitis |
| RG WT B/Bris | ++ | Focal lesions, debris in |
| | | the bronchial lumen, mild |
| | | alveolitis and vasculitis |

Post challenge (5 dpc)

| | | |
|---|---|---|
| Control + | − | — |
| Non-challenged (NC) | | |
| Control + | ++ | Focal lesions, debris in |
| B/Bris PB2-F406Y | | bronchus and bronchioles, |
| | | mild necrosis, alveolitis |
| | | and vasculitis |
| B/Bris att + | − | — |
| B/Bris PB2-F406Y | | |
| Control + | +++ | Focal lesions, much debris |
| B/Wis PB2-F406Y | | in bronchus, presence of |
| | | necrosis and inclusion bodies |
| | | in bronchioles, severe focal |
| | | alveolitis and mild vasculitis |

In order to assess the neutralizing antibody response, all mice were bled at 20 dpi, and HI assays were then performed against the homologous WT B/Bris virus and the heterologous WT B/Wis virus. Nearly all B/Bris att-inoculated mouse serum samples (n=13 out 16) had HI antibody titers equal to or greater than 40 against homologous virus (FIG. 5G), indicating an adequate and potentially protective antibody response on current standards of surrogates of protection. Interestingly, two (n=2 out 16) B/Bris att-inoculated mice showed no seroconversion by HI assay. As expected, none of the B/Bris att-inoculated mice developed HI antibody titers against the heterologous B/Wis virus (FIG. 5G).

### (4) B/Bris att Virus Confers Sterilizing Immunity Against Homologous Challenge (B/Bris PB2-F406Y)

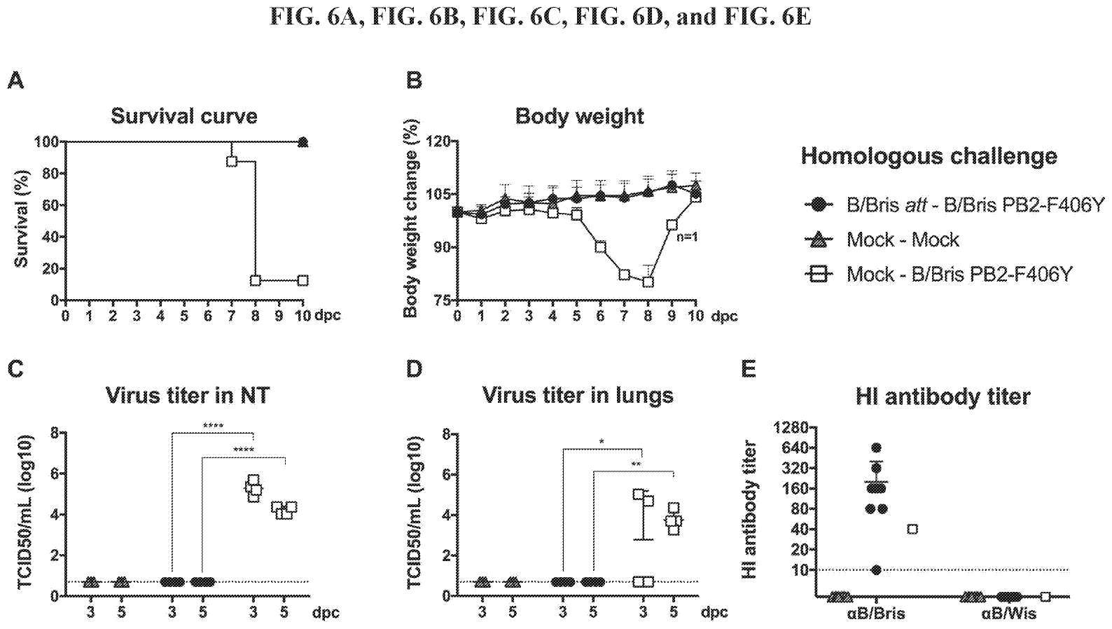

To assess efficacy of the B/Bris att vaccine candidate, mock-vaccinated or B/Bris att-vaccinated mice (n=16/group) were challenged at 21 dpi with 10′ EID₅₀ per mouse of the B/Bris PB2-F406Y virus via the I.N. route. The B/Bris PB2-F406Y virus was chosen over the WT B/Bris because of its increased virulence (FIG. 2). An additional control group remained unchallenged throughout the study (n=16, mock-mock). B/Bris att-vaccinated mice displayed no apparent signs of disease following the B/Bris PB2-F406Y virus challenge. Changes in body weight in the vaccinated/challenge mouse group were indistinguishable from those seen in the unchallenged control group (FIG. 6B). All B/Bris att-vaccinated mice survived challenge with the B/Bris PB2-F406Y virus (P<0.0001, FIG. 6A). In contrast, control mock-vaccinated mice challenged with the B/Bris PB2-F406Y virus experienced extensive clinical signs of disease and body weight loss. With the exception of one mouse, all mock-vaccinated mice challenged with the B/Bris PB2-F406Y virus either succumbed to infection or had to be humanely euthanized by day 8 post-challenge (FIG. 6A). Lung and nasal turbinates were harvested for virus titration from all groups at 3 and 5 dpc. No virus was detected in B/Bris att-vaccinated mice at either 3 or 5 dpc in either the lungs or the nasal turbinates (FIGS. 6C and D). As expected, extensive virus replication in the entire respiratory tract, including both lungs and nasal turbinates, was detected at 3 and 5 dpc in the mock-vaccinated, B/Bris PB2-F406Y-challenged mice (FIGS. 6C and D). In addition, B/Bris att-vaccinated mice, showed an increase in the HI antibody titers against the homologous B/Bris virus at 21 dpc (FIG. 6E). Overall, these results indicate that the B/Bris att vaccine candidate confers sterilizing immunity in mice against the homologous virus.

### (5) B/Bris att Virus Protects Mice Against Heterologous Challenge (B/Wis PB2-F406Y)

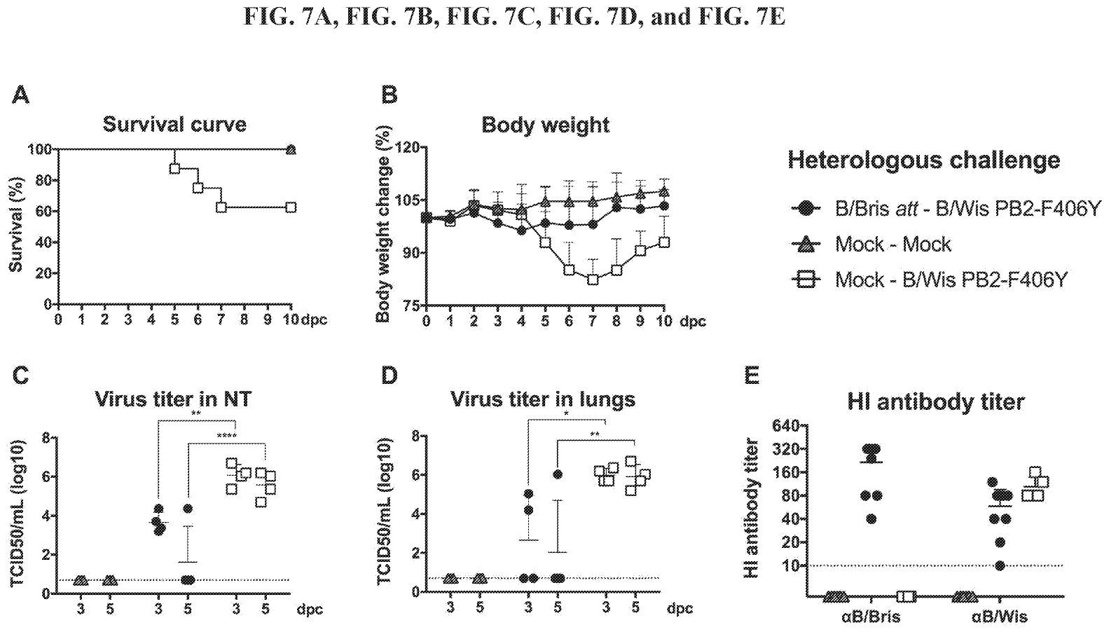

Live attenuated influenza vaccines (LAIV) have been shown to provide more cross-protective immune responses compared to inactivated vaccines by eliciting local mucosal immunity in addition to systemic B and T cell-mediated responses. In order to evaluate the extent of cross-protection, mock- or B/Bris att-vaccinated mice were challenged at 21 dpi with 10⁷ EID₅₀ per mouse of the B/Wis PB2-F406Y virus. All B/Bris att-vaccinated mice (n=8) survived the challenge with the heterologous B/Wis PB2-F406Y virus, whereas ˜40% of challenged mock-vaccinated mice (P<0.05) either succumbed to virus infection or were euthanized due severe clinical disease and/or weight loss (FIGS. 7A and B). In contrast, B/Bris att-vaccinated mice experienced only mild body weight loss following the heterologous challenge (FIG. 7B). At 3 and 5 dpc, analysis of lung (P<0.05 at 3 dpc and P<0.01 at 5 dpc) and nasal turbinate (P<0.01 at 3 dpc and P<0.0001 at 5 dpc) tissues collected showed reduced virus load in B/Bris att-vaccinated mice than in challenged mock-vaccinated mice (FIGS. 7C and D). As expected, the B/Bris att-vaccinated mice experienced a boost in HI antibody titers against the vaccine virus and limited responses to the heterologous B/Wis PB2-F406Y challenge virus when measured at 21 dpc (FIG. 7E). B/Bris att immunization did not provide sterilizing immunity following the heterologous B/Wis PB2-F406Y virus challenge. Nonetheless, nearly all B/Bris att-vaccinated mice [n=3 out 4 (75%)] cleared the challenge virus infection by 5 dpc (FIGS. 7C and D), indicating a cross-protective response mediated by non-neutralizing antibodies, T cells and/or a combination of both.

### (6) B/Bris att Vaccination Reduces Lungs Pathology after IBV Challenge

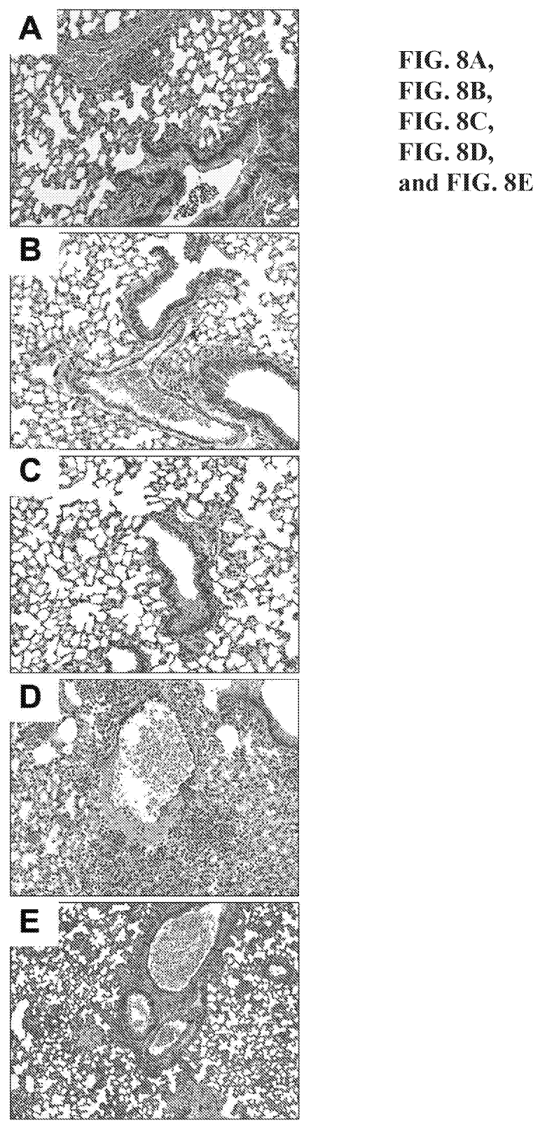

Histopathological examination of lung samples collected at 5 dpc further confirmed the vaccine-challenge studies. Lungs from mock-vaccinated mice challenged with the B/Bris PB2-F406Y virus earned a whole lung score of two (2) with the presence of focal lesions, debris in bronchus and bronchioles, mild necrosis, alveolitis, and vasculitis (Table 2, FIG. 8A). In contrast, lungs from B/Bris att-vaccinated mice challenged with the B/Bris PB2-F406Y virus received an overall histopathology score of zero (0), similar to lungs from mock-vaccinated, mock-challenged mice (Table 2, FIGS. 8B and C). While the B/Wis PB2-F406Y virus was less lethal than the B/Bris PB2-F406Y virus in DBA/2J mice, tissue pathology was more severe. On a 0-4 point scale of lung pathology severity, lungs from mock-vaccinated mice challenged with the B/Wis PB2-F406Y virus obtained a whole lung score of three (3) with the presence of focal lesions, much debris in bronchus, necrosis and inclusion bodies in bronchioles, severe focal alveolitis and mild vasculitis (Table 2, FIG. 8D). Although lungs from B/Bris att-vaccinated mice challenged with B/Wis PB2-F406Y also received a whole lung score of three (3), histopathology findings (debris in bronchus and bronchioles, moderate focal alveolitis and minimal vasculitis) were milder compared to the challenged mock-vaccinated group (Table 2, FIG. 8E). These results are consistent with the protective role of the B/Bris att-stimulated immune response in reducing virus load and promoting rapid virus clearance. Collectively, these results indicate that the B/Bris au vaccine candidate induces a robust protective immune response that can reduce lung pathology in mice following challenge with either antigenically homologous or heterologous IBVs.

### (7) Stability of B/Bris att Virus

Ascertaining genetic stability remains an essential step in the development of LAIV candidates. Thus, the B/Bris au virus from the E2 stock was serially passaged at 33° C. in either in SPF eggs (15 times, sE1 through sE15) or in MDCK cells (20 times, sP1 through sP20). Following the serial passage, isolated RNA from both allantoic fluid and tissue culture supernatant was submitted to whole IBV genome amplification and next-generation sequencing. Sequencing analysis of the PB1 segment confirmed that all modifications introduced (E580G, S660A, and modified HA tag) were stably maintained following successive passages in both SPF eggs and/or MDCK cells (Table 3). In PB1, one mutation at the amino acid position E48K (G163A) emerged in sP1 and was maintained through sP20. Interestingly, both sP1 and sP20 viruses contained the PB1 K391E mutation, which was previously lost (or at least below limit of detection) during preparation of the E2 stock. It is possible that a small virus population remained in the E2 virus stock containing the PB1 K391E mutation. It is tempting to speculate that the E391 becomes stabilized in the MDCK cell passage virus with the acquisition of the potentially compensatory PB1 E48K mutation. The sE15 passage virus showed the WT PB1 sequences at both of these positions (E48 and K391); however, it showed one change from the E2 virus stock at the position V474I (G1441A) (Table 3). The vRNA extracted from pooled nasal turbinate tissues of B/Bris att-vaccinated mice at 3 and 5 dpi revealed the stability of the inserted attenuation markers (E580G, S660A, and modified HA tag). More importantly, no additional mutations were found within the sequenced PB1 segment from nasal turbinate samples. These results emphasize the safety and stability of the B/Bris au virus in vivo.

TABLE 3
Comparison of amino acid substitutions in Flu-B att strains

| | | | | | | | | | | Cold- | |
|---|---|---|---|---|---|---|---|---|---|---|---|
| | | B/Bris | B/Bris | | | | | | | adapted | Cold- |
| Flu-B | | att | att | | B/Bris att | B/Bris att | B/Bris att | B/Bris att | B/Bris att | B/Ann | adapted |
| virus | B/Bris att | MDCK | MDCK | B/Bris att | Egg | MDCK | MDCK | Egg | Egg | Arbor/1/ | B/USSR/ |
| protein | Predicted | (P1) | (P2) | Egg (E1) | (E2) | (sP1) | (sP20) | (sE1) | (sE15) | 1966* | 60/1969ⁱ |
| PB2 | None | NT | NT | NT | NT | None | None | None | S764 | R78Q | 3 mutations |
| | | | | | | | | | (T2315C)^{Syn} | M183I | |
| | | | | | | | | | | V269I | |
| | | | | | | | | | | S630R | |
| PB1 | E391 | E391 | E391 | E391K | E391K | E48K | E48K | E391K | E391K | R433K | |
| | (G1192) | (G1192) | (G1192) | (G1192A)^{Rev} | (G1192A)^{Rev} | (G163A)^{NonS} | (G163A)^{NonS} | (G1192A)^{Rev} | (G1192A)^{Rev} | | |
| | G580 | G580 | G580 | G580 | G580 | E391 | E391 | G580 | V4741 | I651V | |
| | (G1760) | (G1760) | (G1760) | (G1760) | (G1760) | (G1192) | (G1192) | (G1760) | (G1441A)^{NonS} | | |
| | A660 | A660 | A660 | A660 | A660 | G580 | G580 | A660 | G580 | H751Y | |
| | (G1999) | (G1999) | (G1999) | (G1999) | (G1999) | (G1760) | (G1760) | (G1999) | (G1760) | | |
| | HA tag | HA tag | HA tag | HA tag | HA tag | A660 | A660 | HA tag | A660 | | |
| | | | | | | (G1999) | (G1999) | | (G1999) | | |
| | | | | | | HA tag | HA tag | | HA tag | | |
| PA | None | NT | NT | NT | NT | None | E296 | None | K489Q | H160S | 2 mutations |
| | | | | | | | (G917A)Syn | | (A1494C)^{NonS} | S271N | |
| | | | | | | | | | | V431M | |
| | | | | | | | | | | I495M | |
| | | | | | | | | | | Y497H | |
| | | | | | | | | | | D589E | |
| HA | None | NT | NT | NT | NT | None | None | None | None | NT | |
| NP | None | NT | NT | NT | NT | None | K341 | None | D377N | T55A | |
| | | | | | | | (A1083G)^{Syn} | | (G1189A)^{NonS} | I61D | |
| | | | | | | | | | | Y129F | |
| | | | | | | | | | | V114A | |
| | | | | | | | | | | P410H | |
| | | | | | | | | | | A509T | |
| | | | | | | | | | | I531T | |
| | | | | | | | | | | V534I | |
| | | | | | | | | | | D535E | |
| NA | None | NT | NT | NT | NT | None | K371E | None | None | NT | |
| | | | | | | | (A1164G)^{Non} | | | | |
| | | | | | | | S | | | | |
| NB | None | NT | NT | NT | NT | None | None | None | None | NT | |
| BM1 | None | NT | NT | NT | NT | None | None | None | None | H159Q | 1 mutation |
| | | | | | | | | | | M183V | |
| BM2 | None | NT | NT | NT | NT | None | None | None | None | None | 1 mutation |
| NS1 | None | NT | NT | NT | NT | None | M106T | None | None | None | 1 mutation |
| | | | | | | | (T361C)^{NonS} | | | | |
| NEP | None | NT | NT | NT | NT | None | None | None | None | None | |

NT, not tested.
*Adapted from DeBorde DC et al. 1988. Sequence comparison of wild-type and cold-adapted B/Ann Arbor/1/66 influenza virus genes. Virology 163:429-443.
#Adapted from Kiseleva IV et al. 2010. PB2 and PA genes control the expression of the temperature-sensitive phenotype of cold-adapted B/USSR/60/69 influenza master donor virus. J Gen Virol
91(4):931-937 (sequence information not publically available, only the number of mutations per segment).
NonS: Non synonymous mutation, amino acid change
Rev: Reversion to wild type amino acid sequence
Syn: Synonymous mutation, no amino acid change

### c) Discussion

This study described the development of an IBV vaccine candidate with a modified PB1 segment, B/Bris att. The selection of mutations in PB2 and PB1 and incorporation of the HA epitope tag in PB1 were based on experience with IAV att virus vaccines and the assumption that the analogous amino acids in IBV overlap regions with similar function. The original goal was to incorporate all possible analogous mutations; however, a limited set of those were either tolerated, stable, or provided the desired attenuated phenotype. The PB2 K267S (N265S in the IAV PB2) was deleterious for virus rescue but not for polymerase activity, which was reduced in the context of PB1 att modifications, in minigenome assays. Surprisingly, alternative mutations in PB2, previously known to impair cap-binding activity (F406Y or W359F), did not attenuate but instead increased B/Bris virulence in mice compared to the WT strain. It is well established that in mice, IBV strains are usually not lethal unless the virus is forcibly adapted in this species. Advantage was taken of these new findings and used the B/Bris PB2-F406Y virus for lethal challenge in subsequent vaccine efficacy studies. Similarly, the B/Wis PB2-F406Y virus was created for the heterologous challenge, which was more lethal to mice than the WT B/Wis virus but less than the B/Bris PB2-F406Y virus.

Sanger-based and next generation sequencing was utilized to better understand the stability of the B/Bris att virus within and beyond the engineered mutations and HA tag addition. Whole genome sequences of the early passages of the B/Bris att virus obtained from SPF eggs or tissue culture cells were compared to those obtained by passaging the virus multiple times in either SPF eggs or MDCK cells. These sequences were further compared to IBV PB1 sequences obtained from the Influenza Research Database. More than 4,000 complete IBV PB1 sequences are available in IRD, but only 793 differ from each other in at least one amino acid, demonstrating remarkable stability for this particular segment.

The PB1 K391E and E580G mutations (same as in IAV PB1) were tolerated and resulted in adequate polymerase activity; however, the K391E was not always stably maintained in SPF eggs. The K391E mutation was present in virus grown in MDCK cells (P1) but it was lost upon passage in SPF eggs (E2). The emergence of the PB1 E48K mutation played a compensatory role and contributed to maintaining the K391E mutation in MDCK cells. It must be noted that the PB1 E48K mutation emerged also in an alternative virus rescue iteration of B/Bris is (no HA tag) that maintained also the K391E mutation after nine serial passages in eggs (sE9). The PB1 E48K mutation is unique to the recombinant virus prepared in this report, no other IBV virus PB1 segment appears to favor such mutation in nature. 97. The PB1 E580G was stably maintained in MDCK cells, in SPF eggs, and after replication for at least 5 days in mice. The PB1 E580G mutation is not unique to the B/Bris att virus, although it does not appear to be highly favored in nature: Only 4 out of the 793 unique IBV PB1 sequences analyzed contained G580, ˜0.5% if all non duplicated IBV PB1 sequences available in IRD are considered. No association of the PB1 G580 mutant strains with any particular season or location was found; they represent isolates co-circulating with the more favored PB1 E580 (wild type) strains.

The design of the PB1 S660A mutation (A661T in IAV PB1) was counterintuitive. In IAV, the hydrophobic to polar amino acid change in the PB1A661T mutation contributes to its attenuation. It was simply assumed that the opposite change in IBV PB1, the polar to hydrophobic amino acid S660A mutation, would be stable and contribute to virus' attenuation. In fact, the S660A mutation, unique to the B/Bris att virus, was stable over multiple serial passages in either SPF eggs or MDCK cells and contributed to the ts phenotype of B/Bris.

The incorporation of the C-terminal HA tag in PB1 of B/Bris was also well tolerated and remarkably stable over multiple serial passages in either SPF eggs or MDCK cells. Just like in the modified IAV viruses, the HA tag does not severely affect IBV's polymerase activity and it is not sufficient to attenuate the virus in vivo. The combination of E580G and S660A mutations with the C-terminal HA tag in PB1 yielded the B/Bris att virus with a ts phenotype in vitro and attenuation in vivo. The ts phenotype was evident by reduced polymerase complex activity and decreased virus growth kinetics at elevated temperatures (>37° C.). The defective replication of the B/Bris att virus in the lungs of mice indicates that the virus is attenuated in vivo. The PB1 E580G, S660A, and HA tag were not only stably maintained in either SPF eggs or MDCK cells after multiple passages but also for at least 5 days in virus isolated from nasal turbinates of infected mice.

The sE15 passage B/Bris att virus showed one additional amino acid mutation in PB1, V474I. The presence of PB1 1474 is fairly common among field IBV isolates. Neither V474 nor 1474 is fixed in the IBV PB1 virus population. The PB1 1474 is present in 100% of strains isolated from 1940 until 2000, including the cold-adapted B/Ann Arbor/1/66 strain. Therefore, it is safe to speculate that the V474I mutation is unlikely to affect the in vitro or in vivo phenotype of B/Bris att. It remains to be determined whether the V474I mutation reflects egg-grown adaptation. In addition, in the sE15 passage virus, single amino acid mutations were found in PA (K489Q) and NP (D377N). The PA K489Q mutation appears to be unique to the sE15 B/Bris att virus, whereas the NP D377N is found in at least one field isolate. The effect of these mutations on virus attenuation is unknown. Other segments remained unchanged, neither amino acid nor nucleotide changes were observed in the sE15 virus compared to the predicted wild type sequence. Likewise, no amino acid changes were observed in the sP20 virus for the PB2, PA, HA, NP, NB, M, BM, and NEP open reading frames compared to predicted sequences. The sP20 virus did show two amino acid changes with respect to the sP1 virus, one in NA (K371E) and one in NS1 (M106T). Both of these mutations are not unique to the sP20 virus, the NA K371E mutation is fairly common among IBV strains. Next generation sequencing allows for an unprecedented level of detail to examine live virus vaccine stability. Although amino acid changes were observed upon serial passage, the B/Bris att virus showed no changes in HA and great stability at the engineered sites in PB1. Other changes do not appear critical to change the virus' attenuated phenotype.

The growth characteristics of the B/Bris att virus are ideal for a safe LAIV vaccine. The B/Bris att was safe in mice causing no weight loss following inoculation with a high virus dose of 10⁶ EID₅₀. Virus replication was restricted to the upper respiratory tract, as evidenced by virus detection in nasal turbinates but not in lungs. Upon challenge with the homologous B/Bris PB2 F409Y strain, all B/Bris att-vaccinated mice showed sterilizing immunity as no challenge virus was detected in either the lungs or nasal turbinates on any of the days surveyed. Furthermore, no weight loss was seen in any vaccinated mice post-challenge. In contrast, challenged mice in the mock-vaccinated group succumbed to infection; except for one surviving mouse, which nevertheless experienced approximately 20% weight loss post-challenge.

B/Bris att-vaccinated mice also survived the challenge with the heterologous B/Wis PB2-F406Y strain. Although the B/Bris att-vaccinated mice experienced some mild weight loss following challenge with the heterologous virus, this was in sharp contrast to the challenged mock-vaccinated mouse group, which showed significant body weight loss. The B/Bris au protective responses did not confer sterilizing immunity against heterologous challenge. However, most B B/Bris att-vaccinated mice generated an adequate immune response to reduce virus replication and promote rapid viral clearance. Local mucosal immunity is likely involved in the mechanism of protection due the lack of cross-reactivity in the HI antibody response among antigenically distinct IBV HA lineages. More recently, several studies have demonstrated the development of tissue-resident memory (T_{RM}) cells in the lungs after influenza infection and their critical role in enhanced protection against infection at peripheral entry sites. Further studies must be pursued in order to better define the humoral as well as T cell dependent responses to the B/Bris au vaccine that contribute to cross-protection.

In the past few years, influenza vaccine development has shifted to finding universal vaccine approaches that will require fewer updates and provide longer lasting immunity. For the most part, these efforts have overlooked the potential benefits of developing universal vaccines around the concept of a live attenuated virus and have largely focused on eliciting broadly neutralizing antibody responses to more conserved regions of the HA. While recent advances in antigen design to break immunodominance of HA head and induce broadly protective responses against the HA stalk have been made, sequential exposure in the human population to circulating seasonal influenza strains may undercut the long term feasibility of these strategies, as a recent study has demonstrated. Live-attenuated vaccines have been shown to confer better protection than inactivated vaccines due to the stimulation of both humoral (primarily IgG and IgA) and T-cell (virus specific CD4+ and CD8+ T cells) mediated immune responses, instead of simply the humoral (IgG) response associated with inactivated and some universal vaccines (54-56). LAIV vaccines have also been shown to heighten innate immune responses and stimulate cross-protective responses to heterologous or antigenically divergent strains. Other advantages include straightforward administration of LAIV vaccines (needle-free delivery) and smaller infrastructure capacity for manufacturing and processing of LAIV vaccines.

The licensed LAIV vaccine in the United States (FluMist) has suffered a number of issues in vaccine effectiveness over the past three seasons (2013-2014, 2014-2015, and 2015-2016). While the reasons for the limited robustness of vaccine-elicited immune response remains controversial and a matter of ongoing investigation, possible culprits include the suboptimal performance of the H1N1 component, the inclusion of a second IBV strain and possibly reduced immunogenicity due to sequential exposure to the same LAIV vaccine backbones over the years. These perceived limitations of the currently licensed LAIV vaccine based on A/Ann Arbor/6/60 (H2N2) and B/Ann Arbor/1/66 cold-adapted (ca) backbones highlight the critical importance of continued investment in LAIV development and open up the opportunity to improve the current strategy in order to develop more efficacious vaccines. A variety of strategies have been employed to develop alternative experimental LAIV influenza vaccines. Such strategies include, but are not limited to, whole and partial gene knockouts, the insertion of foreign sequences, and the manipulation of the HA cleavage site. Extensive work has been done on NS, M, NA and PB2 partial and full knockout vaccines, and while such vaccines have proven effective, there are drawbacks. For instance, the full knockout vaccines must be grown in cell lines which are not FDA approved that stably express the missing gene in order to achieve the level of growth required of a vaccine strain. The manipulated HA cleavage site strategy, which has been shown to limit virus growth to the presence of elastase, grows to high titers in approved cell lines but has shown some signs of instability in vitro. Finally, while some of these strategies have been shown to be effective in the context of IBV, most of these strategies have only been tested in the context of IAV.

The laboratory has developed an alternative IAV strategy, which incorporates the PB2 and PB1 mutations found in the A/Ann Arbor cold-adapted backbone. Additionally, the strategy involves an in-frame introduction of a 9-amino acid HA tag derived from H3 HA at the C-terminus of PB1. The safety and efficacy of the strategy has been demonstrated in ovo as well as in mouse, chicken, and pig models. Furthermore, it was shown that it grows to high titers in established cell lines and SPF eggs. The current study shows that 2 mutations, rather than the 7 mutations found in the licensed B/Ann Arbor ca backbone, in combination with a modified HA tag at the C-terminus of PB1 are sufficient to attenuate IBV in the context of a mouse model. Notably, the 2 mutations incorporated into the attenuated IBV virus (E580G and S660A) are analogous to a subset of the mutations present in the A/Ann Arbor ca backbone. The availability of contemporary attenuated IAV and IBV (this study) backbones offers an alternative platform for the development of LAIVs that can overcome current limitations.

### F. EXAMPLE 2: LIVE ATTENUATED INFLUENZA VIRUS QUADRIVALENT VACCINE

To test the live attenuated influenza B viruses in a formulation that is more relevant to influenza virus protection, a quadrivalent was made to provide protection to both Influenza A and Influenza B. The quadrivalent influenza virus (QIV) formulation comprised Ty/04 att (H3N2), ma Ca/04 att (H1N1), B/Bris att and B/Wisc att @ 10⁶ TCID₅₀ each virus/50 μl.

### 1. Methods

### a) QIV Vaccine Formulation

Ty/04 att (H3N2), ma Ca/04 att (H1N1), B/Bris att and B/Wisc att @ 10\hat{ }6 TCID50 each virus/50 μl were used to immunize mice. The Ty/04 att (H3N2) strain contains 6 gene segments from wild type A/turkey/OH/313053/2004 (H3N2) virus and PB2att and PB1 att gene segments. The ma Ca/04 (H1N1) att strain contains 6 gene segments from mouse-adapted A/California/04/09 (H1N1) and PB2att and PB1 att gene segments from Ty/04 att (H3N2). The B/Bris att contains 7 gene segments from wild type B/Brisbane/60/2008 and the PB1att segment (attenuation at residue 580 and/or 660 of the PB1 segment of the viral polymerase). The B/Wis att contains the surface gene segments from B/Wisconsin/01/2010 in the background of B/Bris att.

### b) Challenge Viruses

Viruses used in the mouse challenges were the ma Ca/04 (H1N1) strain and the B/Bris PB2 F406Y strain. The ma Ca/04 (H1N1) strain is the mouse-adapted A/California/04/09 (H1N1) strain described in Ye et al, PLoS Path.2010 obtained after a single passage of the wild type Ca/04 (H1N1) virus in DBA/J2 mice. The B/Bris PB2 F406Y is a strain derived from B/Brisbane/60/2008 carrying a mutation in PB2 (F406Y) that increases its virulence in mice as previously described in Santos et al JVI 2017.

### c) Immunization and Challenge

Mice were immunized using an initial priming immunization at day 0 with the QIV delivered intranasally followed by a boost immunization at day 21 with the QIV via intranasal inoculation. 42 days following initial immunization, mice were challenged with 10⁷ TCID₅₀/virus/mouse via intranasal inoculation with either the ma Ca/04 (H1N1) strain (Influenza A) or the B/Bris PB2 F406Y strain (Influenza B). Mice were bled at day 20 prior to the boost with QIV and again at day 41 prior to challenge. Following the challenge, mice were sacrificed at day 4 post challenge (day 47 post initial immunization) and day 21 post challenge (day 63 post initial immunization). Weight loss and survival were measured throughout the immunization and challenge.

### 2. Results

### a) QIV Protects Mice Against Influenza A and B Viruses

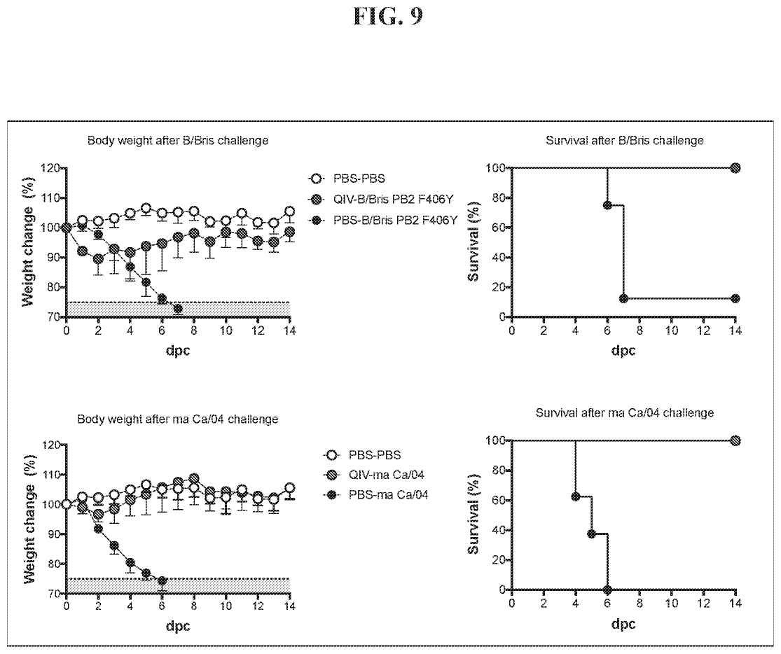

To test whether the QIV vaccine was effective against aggressive challenge with influenza A and B viruses, DBA/2J mice were inoculated with QIV or mock immunized with PBS. At 21 dpi, the mice were boosted using the QIV as explained above. The day before challenge [20 day-post-boost (dpb)], 4 mice/group were bled and sera collected to measure neutralizing antibody responses. At 21 dpb, mice were challenged with either 10⁷ TCID₅₀/mouse of mouse-adapted Ca/04 (ma-Ca/04) (QIV group n=11, mock vaccinated PBS n=11) or with 10⁷ TCID₅₀/mouse B/Bris PB2-F406Y (QIV group n=11, mock vaccinated PBS n=11). A third group was challenged with mock PBS as controls (n=8). The mice were monitored daily for body weight loss and survival. At 5 day-post challenge (dpc), 3 mice per group were sacrificed and lung and nasal turbinate samples were collected for quantitation of virus titers. At 21 dpc, all mice were sacrificed and sera, nasal washes and bronchoalveolar lavage fluids (BALFs) were collected to evaluate antibody responses. As shown in FIG. 9, compared to mock immunized/mock infected negative control groups, mice receiving the QIV did not have any significant weight loss following challenge and had 100% survival throughout the challenge period. By contrast, mock immunized mice receiving challenge showed significant weight loss with no survivors in the influenza A challenge group and only 20% survivability in the Influenza B challenge group.

### b) QIV Prevents Replication of Challenge Virus in Mice

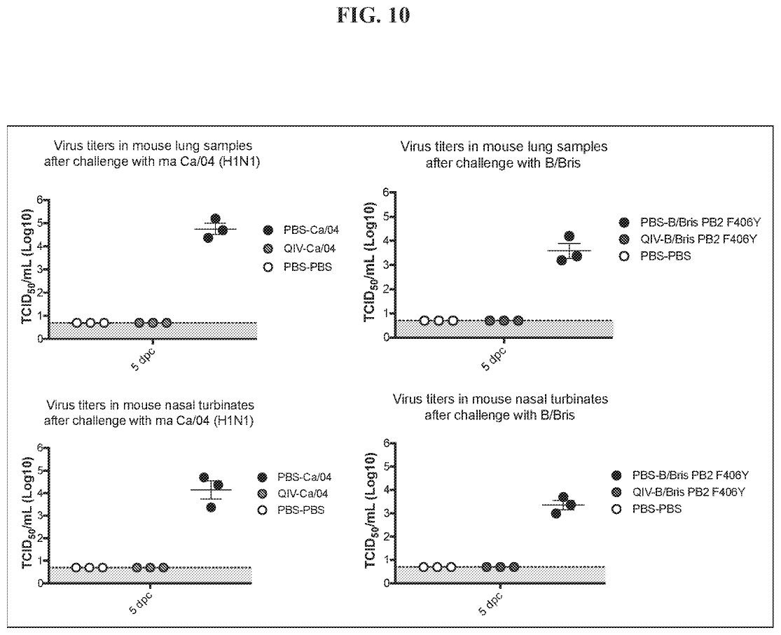

Lung and nasal turbinate samples were collected for titration of virus shedding 5 days post-challenge (FIG. 10). QIV-vaccinated mice showed challenge virus at below limits of detection, whereas PBS-control vaccinated mice showed significant virus titers in both lungs and nasal turbinates after challenge.

### c) QIV Elicits Discernible HI Responses after Boost

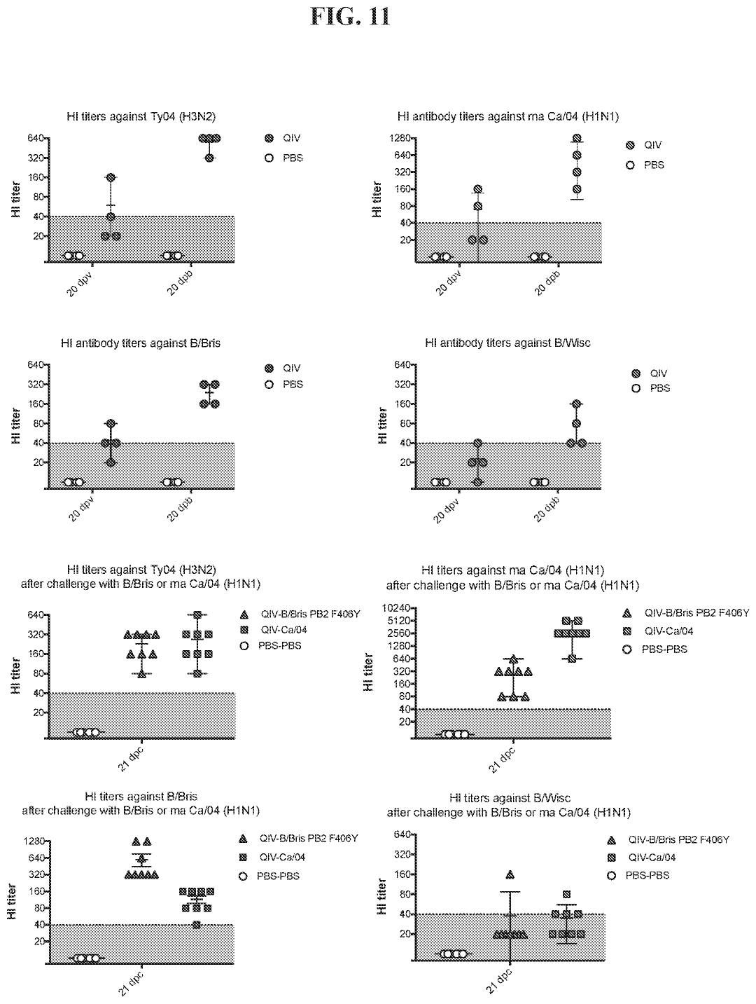

Serum samples collected at 20 dpi, 20 dpb and 21 dpc were analyzed for the presence of neutralizing antibodies by the hemagglutination inhibition (HI) assay using the WT Ty/04 (H3N2), ma Ca/04 (H1N1), WT B/Bris and WT B/Wis viruses (FIG. 11). HI responses were above background after prime vaccination at levels similar or higher than those reported in the literature using alternative live attenuated influenza virus vaccines. HI titers increased after boost with most responses at 1:40 or higher, which is indicative of protective responses. HI activity after challenge showed the expected pattern of boost responses against the challenge virus (either B/Bris or ma Ca/04 (H1N1) with no significance changes against the heterologous viruses, H3N2 or B/Wis). Please note that there is a single PBS control group for prime and boost and a single PBS-PBS challenge control group, which have been included in every corresponding graph for illustration purposes only.

### d) QIV Promotes Viral-Specific IgA and IgG Responses

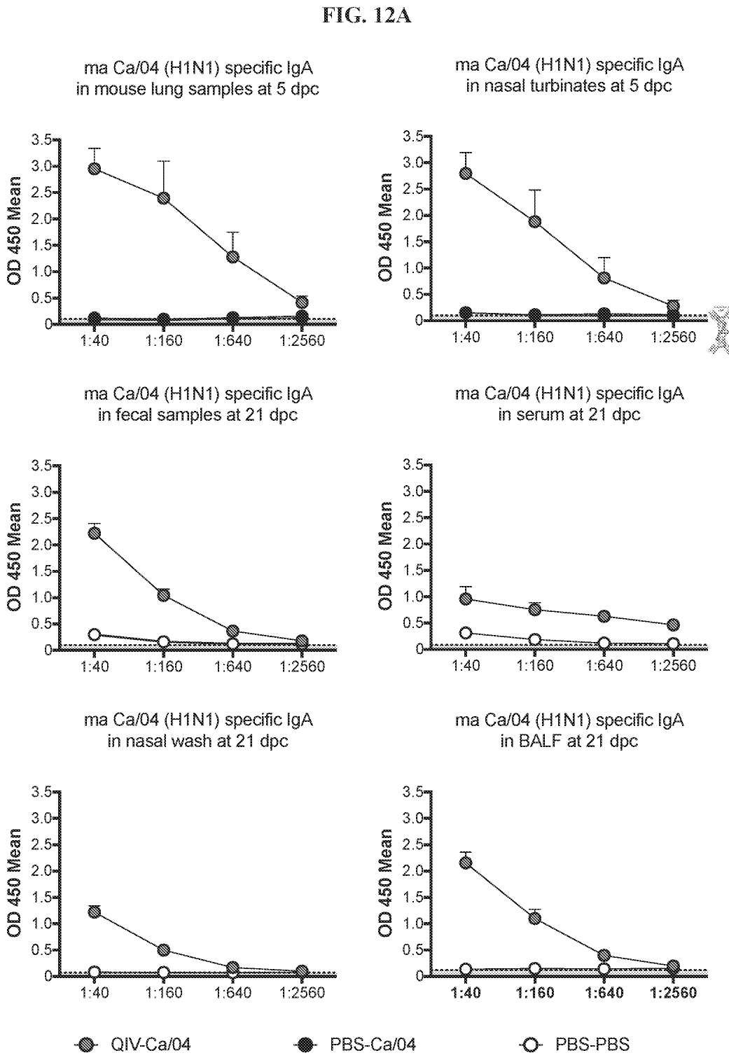

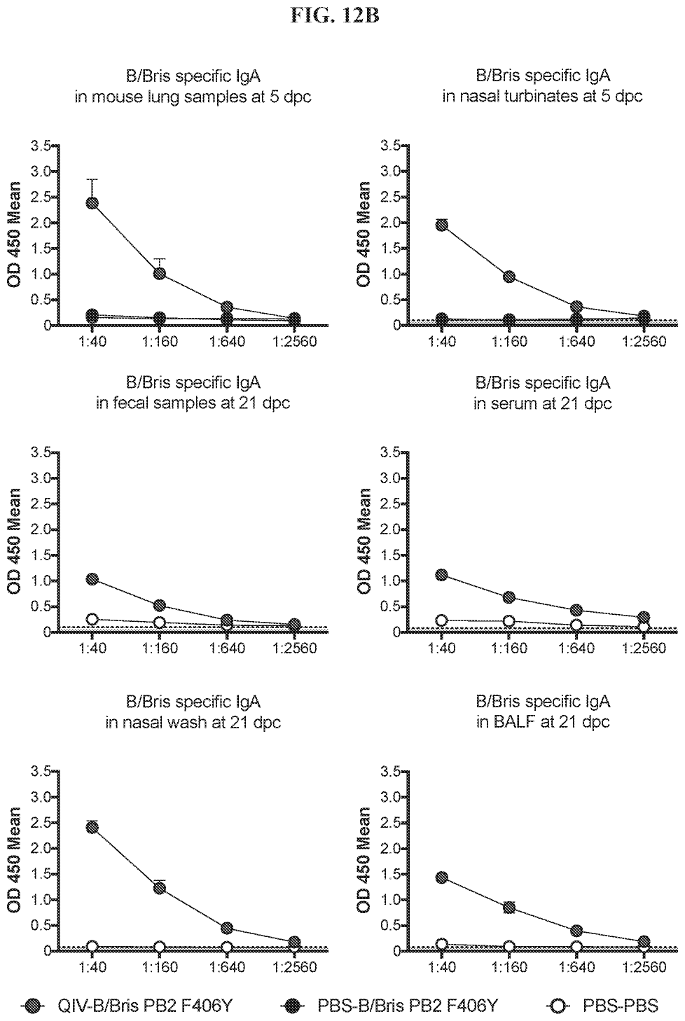

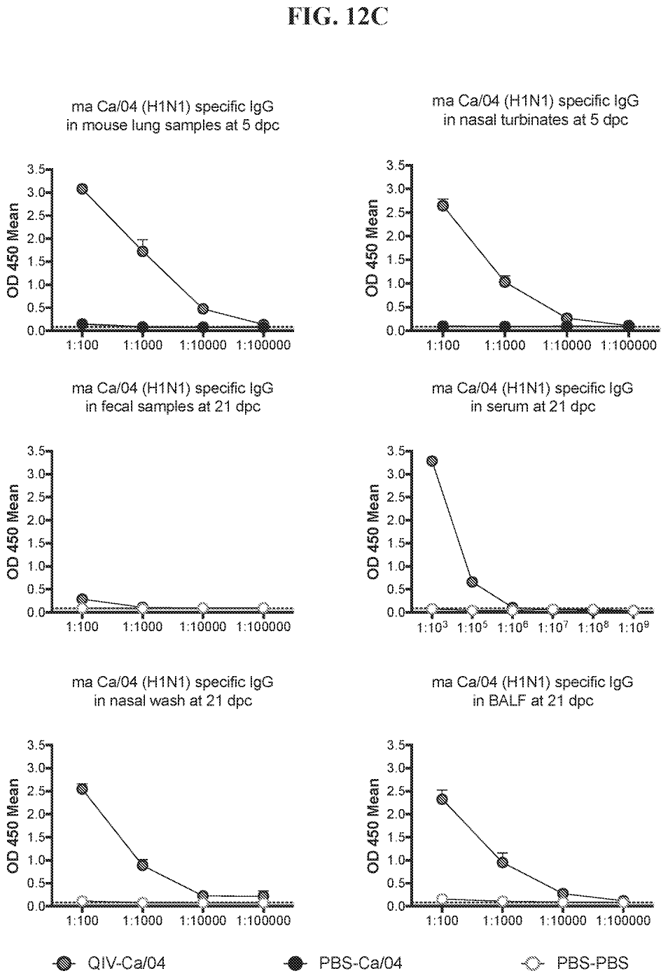

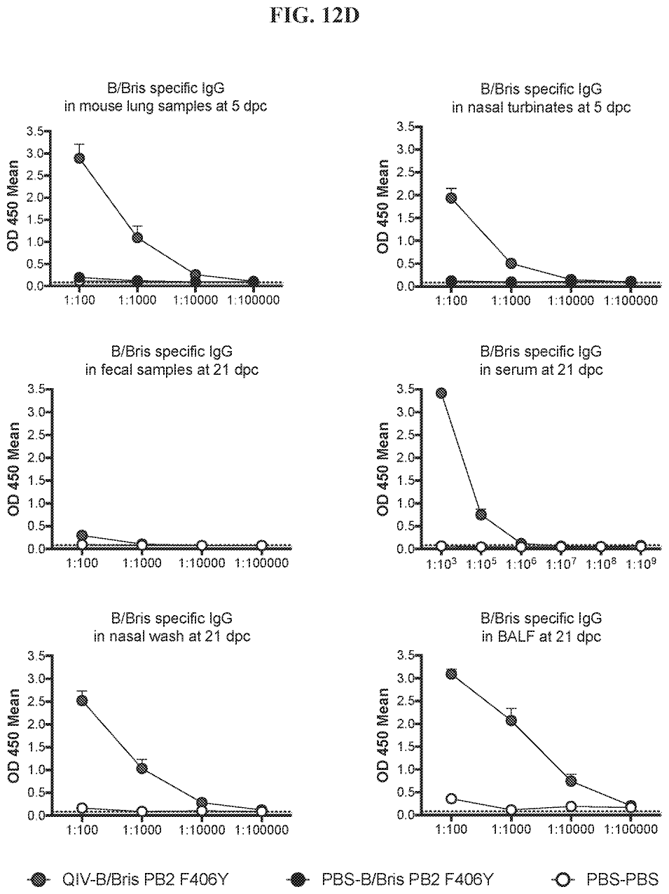

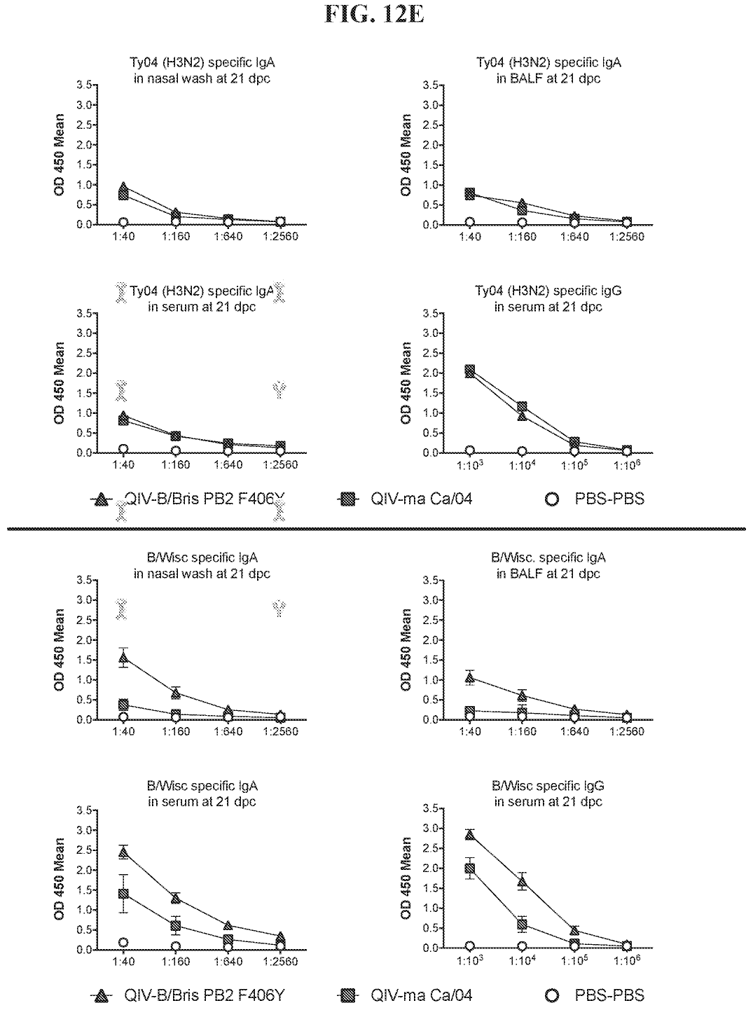

Challenged mice were tested for H1N1-specific IgA responses using an ELISA assay with samples from the lunch and nasal turbinates and samples from feces, sera, nasal washes and collected BALF (FIG. 12A). QIV-vaccinated mice showed high levels of IgA antibody against the ma Ca/04 (H1N1) virus noticeable at 5 dpc (compared to the PBS control challenge with ma Ca/04 (H1N1) that shows no IgA responses). IgA responses are maintained in mice for at least 3 weeks after challenge. Similarly, when assayed from B/Bris specific IgA responses, QIV-vaccinated mice showed high levels of IgA antibody against the B/Bris virus noticeable at 5 dpc (compared to the PBS control challenge with B/Bris that shows no IgA responses) (FIG. 12B). IgA responses are maintained in mice for at least 3 weeks after challenge.

Additionally, when assayed for viral specific IgG antibody, the ELISAs revealed that QIV promotes H1N1-specific IgG responses (FIG. 12C). QIV-vaccinated mice showed high levels of IgG antibody against the ma Ca/04 (H1N1) virus noticeable at 5 dpc (compared to the PBS control challenge with ma Ca/04 (H1N1) that shows no IgG responses). IgG responses are maintained in mice for, at least, 3 weeks after challenge. QIV-vaccinated mice also showed high levels of IgG antibody against the B/Bris virus (FIG. 12D) noticeable at 5 dpc (compared to the PBS control challenge with B/Bris that shows no IgG responses). IgG responses are maintained in mice for, at least, 3 weeks after challenge.

To see if the viral specific IgA and IgG antibodies would also be present for non-challenge H3N2 and B/Wisconsin viruses, ELISAs were run and viral specific antibodies checked. (FIG. 12E). QIV-vaccinated mice showed high levels of IgA and IgG antibody responses against the Ty/04 (H3N2) virus in nasal washes, BALF, and sera collected at 21 dpc with the heterologous ma Ca/04 (H1N1) or B/Bris viruses. Since HI responses did not change against the Ty04 (H3N2) virus after heterologous challenge (FIG. 11), it is reasonable to conclude that Ty04 (H3N2)-specific IgA and IgG responses were elicited by the QIV formulation. Anti-H3N2 IgA and IgG responses were maintained in mice for at least 3 weeks after challenge. At 21 dpc, IgA responses against B/Wisc were not different between the QIV group challenged with the heterologous ma Ca/04 (H1N1) virus and the PBS/non challenge control group. However, IgA responses against the B/Wisc virus were stimulated after challenge with the heterologous B/Bris virus, most likely due to some levels of cross-reactivity. Sera collected at 21 dpc indicate that the QIV formulation was capable of eliciting B/Bris-specific IgA and IgG responses, which were boosted during challenge with the heterologous B/Bris virus.

### 3. Discussion

As shown herein, the QIV formulation in a prime/boost regime was safe and immunogenic and produced HI responses in vaccinated mice against the four corresponding homologous viruses (H3N2, H1N1, and two B strains). Additionally, the QIV produced protective responses against aggressive challenge with either ma-Ca/04 H1N1 or B/Bris viruses. The virus specific IgA and IgG responses were detected against homologous (ma-Ca/04 H1N1 or B/Bris) viruses as early as 5 dpc indicating prior stimulation of such responses by the QIV formulation. In fact, the virus specific IgA and IgG responses were maintained for at least 3 weeks post-challenge. Virus specific IgA and IgG responses were also detected against heterologous (Ty04 H3N2 and B/Wisc) viruses by 21 dpc suggesting that such responses were stimulated by the QIV formulation. Although present, B/Wisc antibody responses were limited. The challenge with B/Bris improved B/Wis-IgA and IgG responses, but not HI responses, probably due to same level of cross-reactivity.

### G. REFERENCES

Alexandrova G I, Maassab H F, Kendal A P, Medvedeva T E, Egorov A Y, Klimov A I, Cox N J. 1990. Laboratory properties of cold-adapted influenza B live vaccine strains developed in the US and USSR, and their B/Ann Arbor/1/86 cold-adapted reassortant vaccine candidates. Vaccine 8:61-64.
Andrews S F, Huang Y, Kaur K, Popova L I, Ho I Y, Pauli N T, Henry Dunand C J, Taylor W M, Lim S, Huang M, Qu X, Lee J H, Salgado-Ferrer M, Krammer F, Palese P, Wrammert J, Ahmed R, Wilson P C. 2015 Immune history profoundly affects broadly protective B cell responses to influenza. Sci Transl Med 7:316ra192.
Baker S F, Nogales A, Finch C, Tuffy K M, Domm W, Perez D R, Topham D J, Martinez-Sobrido L. 2014. Influenza A and B virus intertypic reassortment through compatible viral packaging signals. J Virol 88:10778-10791.
Barr I G, Russell C, Besselaar T G, Cox N J, Daniels R S, Donis R, Engelhardt O G, Grohmann G, Itamura S, Kelso A, McCauley J, Odagiri T, Schultz-Cherry S, Shu Y, Smith D, Tashiro M, Wang D, Webby R, Xu X, Ye Z, Zhang W, Writing Committee of the World Health Organization Consultation on Northern Hemisphere Influenza Vaccine Composition f. 2014. WHO recommendations for the viruses used in the 2013-2014 Northern Hemisphere influenza vaccine: Epidemiology, antigenic and genetic characteristics of influenza A(H1N1)pdm09, A(H3N2) andB influenza viruses collected from October 2012 to January 2013. Vaccine 32:4713-4725.
Briedis D J, Lamb R A. 1982. Influenza B virus genome: sequences and structural organization of RNA segment 8 and the mRNAs coding for the NS1 and NS2 proteins. J 594 Virol 42:186-193.
Camilloni B, Neri M, Lepri E, Basileo M, Sigismondi N, Puzelli S, Donatelli I, Iorio A M. 2010. An influenza B outbreak during the 2007/2008 winter among appropriately immunized elderly people living in a nursing home. Vaccine 28:7536-7541.
Chan W, Zhou H, Kemble G, Jin H. 2008. The cold adapted and temperature sensitive influenza A/Ann Arbor/6/60 virus, the master donor virus for live attenuated influenza vaccines, has multiple defects in replication at the restrictive temperature. Virology 380:304-311.
Chen R, Holmes E C. 2008. The evolutionary dynamics of human influenza B virus. J Mol Evol 66:655-663.
Chen Z, Aspelund A, Kemble G, Jin H. 2006. Genetic mapping of the cold-adapted phenotype of B/Ann Arbor/1/66, the master donor virus for live attenuated influenza vaccines (FluMist). Virology 345:416-423.
Chen Z, Aspelund A, Kemble G, Jin H. 2008. Molecular studies of temperature-sensitive replication of the cold-adapted B/Ann Arbor/1/66, the master donor virus for live attenuated influenza FluMist vaccines. Virology 380:354-362.
Cheng X, Zengel J R, Suguitan A L, Jr., Xu Q, Wang W, Lin J, Jin H. 2013. Evaluation of the humoral and cellular immune responses elicited by the live attenuated and inactivated influenza vaccines and their roles in heterologous protection in ferrets. J Infect Dis 208:594-602.
Daum L T, Canas L C, Klimov A I, Shaw M W, Gibbons R V, Shrestha S K, Myint K S, Acharya R P, Rimal N, Reese F, Niemeyer D M, Arulanandam B P, Chambers J P. 2006. Molecular analysis of isolates from influenza B outbreaks in the U.S. and Nepal, 2005. Arch Virol 151:1863-1874.
DeBorde D C, Donabedian A M, Herlocher M L, Naeve C W, Maassab H F. 1988. Sequence comparison of wild-type and cold-adapted B/Ann Arbor/1/66 influenza virus genes. Virology 163:429-443.
DeBorde D C, Naeve C W, Herlocher M L, Maassab H F. 1987. Nucleotide sequences of viruses. Virus Res 8:33-41.
Della Cioppa G, Vesikari T, Sokal E, Lindert K, Nicolay U. 2011. Trivalent and quadrivalent MF59((R))-adjuvanted influenza vaccine in young children: a dose- and schedule-finding study. Vaccine 29:8696-8704.
Finch C, Li W, Perez D R. 2015. Design of alternative live attenuated influenza virus vaccines. Curr Top Microbiol Immunol 386:205-235.
Gauger P C, Loving C L, Khurana S, Lorusso A, Perez D R, Kehrli M E, Jr., Roth J A, Golding H, Vincent A L. 2014. Live attenuated influenza A virus vaccine protects against A(H1N1)pdm09 heterologous challenge without vaccine associated enhanced respiratory disease. Virology 471-473:93-104.
Grohskopf L A, Sokolow L Z, Broder K R, Olsen S J, Karron R A, Jernigan D B, Bresee J S. 2016. Prevention and Control of Seasonal Influenza with Vaccines. MMWR Recomm Rep 65:1-54.
Hai R, Martinez-Sobrido L, Fraser K A, Ayllon J, Garcia-Sastre A, Palese P. 2008. Influenza B virus NS1-truncated mutants: live-attenuated vaccine approach. J Virol 82:10580-10590.
Hoffmann E, Mahmood K, Chen Z, Yang C F, Spaete J, Greenberg H B, Herlocher M L, Jin H, Kemble G. 2005. Multiple gene segments control the temperature sensitivity and attenuation phenotypes of ca B/Ann Arbor/1/66. J Virol 79:11014-11021.
Hoffmann E, Neumann G, Kawaoka Y, Hobom G, Webster R G. 2000. A DNA transfection system for generation of influenza A virus from eight plasmids. Proc Natl Acad Sci USA 97:6108-6113.
Hoffmann E, Stech J, Guan Y, Webster R G, Perez D R. 2001. Universal primer set for the full-length amplification of all influenza A viruses. Arch Virol 146:2275-2289.
Horvath C M, Williams M A, Lamb R A. 1990. Eukaryotic coupled translation of tandem cistrons: identification of the influenza B virus BM2 polypeptide. EMBO J 9:2639-2647.
Hutchinson E C, Charles P D, Hester S S, Thomas B, Trudgian D, Martinez-Alonso M, Fodor E. 2014. Conserved and host-specific features of influenza virion architecture. Nat Commun 5:4816.
Iijima N, Iwasaki A. 2014. T cell memory. A local macrophage chemokine network sustains protective tissue-resident memory CD4 T cells. Science 346:93-98.
Jain V K, Rivera L, Zaman K, Espos R A, Jr., Sirivichayakul C, Quiambao B P, Rivera-Medina D M, Kerdpanich P, Ceyhan M, Dinleyici E C, Cravioto A, Yunus M, Chanthavanich P, Limkittikul K, Kurugol Z, Alhan E, Caplanusi A, Durviaux S, Boutet P, Ofori-Anyinam O, Chandrasekaran V, Dbaibo G, Innis B L. 2013. Vaccine for prevention of mild and moderate-to-severe influenza in children. N Engl J Med 369:2481-2491.
Jin H, Lu B, Zhou H, Ma C, Zhao J, Yang C F, Kemble G, Greenberg H. 2003. Multiple amino acid residues confer temperature sensitivity to human influenza virus vaccine strains (FluMist) derived from cold-adapted A/Ann Arbor/6/60. Virology 306:18-24.
Jin H, Zhou H, Lu B, Kemble G. 2004. Imparting temperature sensitivity and attenuation in ferrets to A/Puerto Rico/8/34 influenza virus by transferring the genetic signature for temperature sensitivity from cold-adapted A/Ann Arbor/6/60. J Virol 78:995-998.
Krammer F. 2016. Novel universal influenza virus vaccine approaches. Curr Opin Virol 17:95-103.
Laidlaw B J, Decman V, Ali M A, Abt M C, Wolf A I, Monticelli L A, Mozdzanowska K,
Angelosanto J M, Artis D, Erikson J, Wherry E J. 2013. Cooperativity between CD8+ T cells, non-neutralizing antibodies, and alveolar macrophages is important for heterosubtypic influenza virus immunity. PLoS Pathog 9:e1003207.
Laidlaw B J, Zhang N, Marshall H D, Staron M M, Guan T, Hu Y, Cauley L S, Craft J, Kaech S M. 2014. CD4+ T cell help guides formation of CD103+ lung-resident memory CD8+ T cells during influenza viral infection. Immunity 41:633-645.
Lanthier P A, Huston G E, Moquin A, Eaton S M, Szaba F M, Kummer L W, Tighe M P, Kohlmeier J E, Blair P J, Broderick M, Smiley S T, Haynes L. 2011. Live attenuated influenza vaccine (LAIV) impacts innate and adaptive immune responses. Vaccine 29:7849-7856.
Loving C L, Lager K M, Vincent A L, Brockmeier S L, Gauger P C, Anderson T K, Kitikoon P, Perez D R, Kehrli M E, Jr. 2013. Efficacy in pigs of inactivated and live attenuated influenza virus vaccines against infection and transmission of an emerging H3N2 similar to the 2011-2012 H3N2v. J Virol 87:9895-9903.
Loving C L, Vincent A L, Pena L, Perez D R. 2012. Heightened adaptive immune responses following vaccination with a temperature-sensitive, live-attenuated influenza virus compared to adjuvanted, whole-inactivated virus in pigs. Vaccine 30:5830-5838.
Lowen A C, Mubareka S, Steel J, Palese P. 2007. Influenza virus transmission is dependent on relative humidity and temperature. PLoS Pathog 3:1470-1476.
Maassab H F, Francis T, Jr., Davenport F M, Hennessy A V, Minuse E, Anderson G. 1969. Laboratory and clinical characteristics of attenuated strains of influenza virus. Bull World Health Organ 41:589-594.
Maassab H F. 1967. Adaptation and growth characteristics of influenza virus at 25 degrees c. Nature 213:612-614.
McCullers J A, Saito T, Iverson A R. 2004. Multiple genotypes of influenza B virus circulated between 1979 and 2003. J Virol 78:12817-12828.
Mena I, Nelson M I, Quezada-Monroy F, Dutta J, Cortes-Fernandez R, Lara-Puente J H, Castro-Peralta F, Cunha L F, Trovao N S, Lozano-Dubernard B, Rambaut A, van Bakel H, Garcia-Sastre A. 2016. Origins of the 2009 H1N1 influenza pandemic in swine in Mexico. Elife 5.
Monto A S, Miller F D, Maassab H F. 1982. Evaluation of an attenuated, cold-recombinant influenza B virus vaccine. J Infect Dis 145:57-64.
Mosterin Hopping A, Fonville J M, Russell C A, James S, Smith D J. 2016. Influenza B vaccine lineage selection—an optimized trivalent vaccine. Vaccine 34:1617-1622.
Nakaya H I, Wrammert J, Lee E K, Racioppi L, Marie-Kunze S, Haining W N, Means A R, Kasturi S P, Khan N, Li G M, McCausland M, Kanchan V, Kokko K E, Li S, Elbein R, Mehta A K, Aderem A, Subbarao K, Ahmed R, Pulendran B. 2011. Systems biology of vaccination for seasonal influenza in humans. Nat Immunol 12:786-795.
Paul Glezen W, Schmier J K, Kuehn C M, Ryan K J, Oxford J. 2013. The burden of influenza B: a structured literature review. Am J Public Health 103:e43-51.
Pena L, Vincent A L, Ye J, Ciacci-Zanella J R, Angel M, Lorusso A, Gauger P C, Janke B H, Loving C L, Perez D R. 2011. Modifications in the polymerase genes of a swine-like triple-reassortant influenza virus to generate live attenuated vaccines against 2009 pandemic H1N1 viruses. J Virol 85:456-469.
Pica N, Langlois R A, Krammer F, Margine I, Palese P. 2012. NS1-truncated live attenuated virus vaccine provides robust protection to aged mice from viral challenge. J Virol 86:10293-10301.
Reed L J, Muench H. 1938. A simple method for estimating fifty percent endpoints. Am J Hyg 27:493-497.
Ritchey M B, Palese P, Kilbourne E D. 1976. RNAs of influenza A, B, and C viruses. J Virol 18:738-744.
Rota P A, Wallis T R, Harmon M W, Rota J S, Kendal A P, Nerome K. 1990. Cocirculation of two distinct evolutionary lineages of influenza type B virus since 1983. Virology 175:59-68.
Rudenko L, Yeolekar L, Kiseleva I, Isakova-Sivak I. 2016. Development and approval of live attenuated influenza vaccines based on Russian master donor viruses: Process challenges and success stories. Vaccine 34:5436-5441.
Russell C A, Jones T C, Barr I G, Cox N J, Garten R J, Gregory V, Gust I D, Hampson A W, Hay A J, Hurt A C, de Jong J C, Kelso A, Klimov A I, Kageyama T, Komadina N, Lapedes A S, Lin Y P, Mosterin A, Obuchi M, Odagiri T, Osterhaus A D, Rimmelzwaan G F, Shaw M W, Skepner E, Stohr K, Tashiro M, Fouchier R A, Smith D J. 2008. Influenza vaccine strain selection and recent studies on the global migration of seasonal influenza viruses. Vaccine 26 Suppl 4:D31-34.
Seo S U, Byun Y H, Lee E Y, Jung E J, Jang Y H, Kim H A, Ha S H, Lee K H, Seong B L. 2008. Development and characterization of a live attenuated influenza B virus vaccine candidate. Vaccine 26:874-881.
Shaw M W, Choppin P W, Lamb R A. 1983. A previously unrecognized influenza B virus glycoprotein from a bicistronic mRNA that also encodes the viral neuraminidase. Proc Natl Acad Sci USA 80:4879-4883.
Shaw M W, Xu X, Li Y, Normand S, Ueki R T, Kunimoto G Y, Hall H, Klimov A, Cox N J, Subbarao K. 2002. Reappearance and global spread of variants of influenza B/Victoria/2/87 lineage viruses in the 2000-2001 and 2001-2002 seasons. Virology 303:1-8.
Shin H, Iwasaki A. 2013. Tissue-resident memory T cells. Immunol Rev 255:165-181.
Snyder M H, Betts R F, DeBorde D, Tierney E L, Clements M L, Herrington D, Sears S D, Dolin R, Maassab H F, Murphy B R. 1988. Four viral genes independently contribute to attenuation of live influenza A/Ann Arbor/6/60 (H2N2) cold-adapted reassortant virus vaccines. J Virol 62:488-495.
Solorzano A, Ye J, Perez D R. 2010. Alternative live-attenuated influenza vaccines based on modifications in the polymerase genes protect against epidemic and pandemic flu. J Virol 84:4587-4596.
Song H, Nieto G R, Perez D R. 2007. A new generation of modified live-attenuated avian influenza viruses using a two-strategy combination as potential vaccine candidates. J Virol 81:9238-9248.
Stech J, Garn H, Herwig A, Stech O, Dauber B, Wolff T, Mettenleiter T C, Klenk H D. 2011. Influenza B virus with modified hemagglutinin cleavage site as a novel attenuated live vaccine. J Infect Dis 204:1483-1490.
Stech J, Garn H, Wegmann M, Wagner R, Klenk H D. 2005. A new approach to an influenza live vaccine: modification of the cleavage site of hemagglutinin. Nat Med 11:683-689.
Te Velthuis A J, Fodor E. 2016. Influenza virus RNA polymerase: insights into the mechanisms of viral RNA synthesis. Nat Rev Microbiol 14:479-493.
Teij aro J R, Turner D, Pham Q, Wherry E J, Lefrancois L, Farber D L. 2011. Cutting edge: Tissue-retentive lung memory CD4 T cells mediate optimal protection to respiratory virus infection. J Immunol 187:5510-5514.
Thompson W W, Shay D K, Weintraub E, Brammer L, Bridges C B, CoxNJ, Fukuda K. 2004. Influenza-associated hospitalizations in the United States. JAMA 292:1333-1340.
Victor S T, Watanabe S, Katsura H, Ozawa M, Kawaoka Y. 2012. A replication-incompetent PB2-knockout influenza A virus vaccine vector. J Virol 86:4123-4128.
Wakai C, Iwama M, Mizumoto K, Nagata K. 2011. Recognition of cap structure by influenza B virus RNA polymerase is less dependent on the methyl residue than recognition by influenza A virus polymerase. J Virol 85:7504-7512.
Watanabe S, Watanabe T, Kawaoka Y. 2009. Influenza A virus lacking M2 protein as a live attenuated vaccine. J Virol 83:5947-5950.
Win M K, Chow A, Chen M, Lau Y F, Ooi E E, Leo Y S. 2010. Influenza B outbreak among influenza-vaccinated welfare home residents in Singapore. Ann Acad Med Singapore 39:448-452.
Ye J, Sorrell E M, Cai Y, Shao H, Xu K, Pena L, Hickman D, Song H, Angel M, Medina R A, Manicassamy B, Garcia-Sastre A, Perez D R. (2010) Variations in the hemagglutinin of the 2009 H1N1 pandemic virus: potential for strains with altered virulence phenotype? PLoS Pathog. 6(10).
Zhou B, Lin X, Wang W, Halpin R A, Bera J, Stockwell T B, Barr I G, Wentworth D E. 2014. Universal influenza B virus genomic amplification facilitates sequencing, diagnostics, and reverse genetics. J Clin Microbiol 52:1330-1337.
Zhu W, Higgs B W, Morehouse C, Streicher K, Ambrose C S, Woo J, Kemble G W, Jallal B, Yao Y. 2010. A whole genome transcriptional analysis of the early immune response induced by live attenuated and inactivated influenza vaccines in young children. Vaccine 28:2865-2876.

H. Sequences

IAYPYDVPDY (SEQ ID NO: 1)
YPYDVPDY (SEQ ID NO: 2)

1. A live attenuated influenza B virus comprising a substitution at residue 580 or 660 of the PB1 segment of the viral polymerase.

2. The live attenuated Influenza B viruses of claim 1, wherein the substitution at residue 580 is an E580G substitution.

3. The live attenuated Influenza B viruses of claim 1, wherein the substitution at residue 660 is a S660A substation.

4. The live attenuated Influenza B viruses of claim 1, comprising an E580G and a S660A substitution in the PB1 segment of the influenza viral polymerase.

5. The live attenuated Influenza B viruses of claim 1 further comprising a HA-tag.

6. A live attenuated influenza vaccine comprising one or more of the live attenuated influenza viruses of claim 1.

7. The live attenuated influenza vaccine of claim 6, further comprising one or more live attenuated influenza A viruses.

8. A method of inhibiting an Influenza B virus infection comprising administering to a subject a live attenuated Influenza B virus of claim 1.

9. A method of inhibiting an Influenza B virus infection comprising administering to a subject a live attenuated Influenza B virus comprising a substitution of the at residue 580 or 660 of the PB1 segment of the viral polymerase.

10. The method of inhibiting an Influenza B virus infection of claim 9, wherein the substitution at residue 580 of the PB1 segment of the viral polymerase in the attenuated influenza B virus is an E580G substitution.

11. The method of inhibiting an Influenza B virus infection of claim 9, wherein the substitution at residue 660 of the PB1 segment of the viral polymerase in the attenuated influenza B virus is an S660A substitution.

12. The method of inhibiting an Influenza B virus infection of claim 9, wherein the live attenuated Influenza B virus comprises E580G and a S660A substitution in the PB1 segment of the influenza viral polymerase.

13. The method of claim 9, wherein the live attenuated Influenza B virus further comprises an HA-tag.

14. The method of inhibiting an Influenza B virus infection of claim 9, wherein the live attenuated Influenza B virus is administered as a component of a vaccine.

15. The method of inhibiting an Influenza B virus infection of claim 14, wherein the vaccine is a multivalent vaccine.

16. The method of inhibiting an Influenza B virus infection of claim 15, wherein the multivalent vaccine comprises at least two Yamagata lineage influenza B viruses; at least two Victoria lineage influenza B viruses, or at least one Yamagata lineage influenza B virus and at least one Victoria influenza B virus.

17. The method of inhibiting an Influenza B virus infection of any of claim 15 or 16 wherein the multivalent vaccine further comprises one or more live attenuated influenza A viruses.

18. A method of attenuating an influenza B virus comprising substituting the amino acid at residue 580 or 660 of the PB1 segment of the viral polymerase.

19. A method of inhibiting an Influenza B virus infection comprising administering to a subject a vaccine of claim 6.
